(19) United States
(12) Patent Application Publication
(10) Pub. No.: US 2015/0132289 A1
(43) Pub. Date: May 14, 2015

(54) METHODS AND COMPOSITIONS FOR TREATING PSC (PRIMARY SCLEROSING CHOLANGITIS) OR PBC (PRIMARY BILIARY CIRRHOSIS) WITH ANTI-CD3 IMMUNE MOLECULE THERAPY

(71) Applicant: Therapix Biosciences Ltd., Ness Ziona (IL)

(72) Inventors: Ronald Ellis, Jerusalem (IL); Yaron Ilan, Jerusalem (IL); Nadya Lisovoder, Netanya (IL); Shahar Dotan, Omer (IL)

(21) Appl. No.: 14/381,585

(22) PCT Filed: Feb. 23, 2013
(86) PCT No.: PCT/IL2013/050158

§ 371 Date: Aug. 27, 2014

Related U.S. Application Data
(60) Provisional application No. 61/604,853, filed on Feb. 29, 2012.

(51) Int. Cl. C07K 16/28

(52) U.S. Cl. CPC C07K 16/2809; A61K 2039/505
USPC 424/133.1; 424/172.1

(57) ABSTRACT

A method or composition comprising an anti-CD3 immune molecule for treatment of PSC (primary sclerosing cholangitis) or PBC (primary biliary cirrhosis) in a subject.

### FIELD OF THE INVENTION

The present invention relates to methods and compositions for treating PSC or PBC, and in particular, for treating PSC (primary sclerosing cholangitis) or PBC (Primary biliary cirrhosis) with anti-CD3 immune molecules, such as antibodies, which may be administered orally or mucosally.

### BACKGROUND OF THE INVENTION

Immunotherapy strategies that involve antibody-induced signaling through antigen-specific T-cell receptors (TCR) have been shown to ameliorate autoimmune and inflammatory diseases, probably by regulating the immune response to self-antigens. One example of such a receptor is CD3 (cluster of differentiation 3). Parenterally administered anti-CD3 monoclonal antibody (mAb) therapy in particular has been shown to be efficacious in preventing and reversing the onset of diabetes in NOD mice (Chatenoud et al., J. Immunol. 158:2947-54, 1997); Belghith et al., Nat. Med. 9:1202-8, 2003) and in treating subjects with Type 1 diabetes (Herold et al., N. Engl. J. Med. 346:1692-8, 2002), and to reverse experimental allergic encephalomyelitis (EAE) in Lewis rats with a preferential suppressive effect on T-helper type 1 (Th1) cells, which participate in cell-mediated immunity (Tran et al., Intl. Immunol. 13:1109-20; 2001). The FDA approved Orthoclone OKT3 (muromonab-CD3; Ortho Biotech Products, Bridgewater, N.J.), a murine anti-CD3 mAb, for intravenous injection for the treatment of graft rejection after transplantation (Chatenoud, Nat. Rev. Immunol. 3:123-32; 2003).

As described in U.S. Pat. No. 7,883,703 to Howard Weiner et al., which is hereby incorporated by reference as if fully set forth herein, anti-CD3 antibodies are also useful for treatment of autoimmune diseases when administered orally or mucosally. Without wishing to be limited by a single hypothesis, the success of such oral or mucosal administration is attributed to activation of regulatory T cells (Tregs) in the mucosal immune system, which in turn leads to an amelioration or down-regulation of the undesired immune system effects, hence ameliorating or at least reducing the pathology of the autoimmune and inflammatory disease. Among the advantages of the oral or mucosal route over the systemic route of administration of anti-CD3 mAb is the ability to avoid the serious adverse events and generalized immune-suppression associated with systemic administration. This route of administration also acts to increase Tregs and to suppress effector cells.

### SUMMARY OF THE INVENTION

The present invention, in at least some embodiments, provides methods and compositions for treatment of PSC or PBC with anti-CD3 oral or mucosal immune molecule therapy. As used herein, the term “treatment” of PSC or PBC also encompasses preventing progression and/or delaying development of PSC or PBC. As used herein the term “preventing” does not require 100% effectiveness.

“Oral or mucosal immune molecule therapy” means the administration of an active anti-CD3 immune molecule orally or to a mucosal membrane (or a combination thereof). Such an anti-CD3 immune molecule may optionally and preferably comprise an anti-CD3 antibody, for example and without limitation, whole antibodies or active fragments thereof (e.g., F(ab′)₂ or scFv, etc) that can bind specifically to CD3. For the purpose of description only and without wishing to be limited in any way, reference may be made herein to an anti-CD3 antibody; it is understood that such a reference may refer to any anti-CD3 immune molecule that is suitable for oral or mucosal administration.

The term “PSC” refers to primary sclerosing cholangitis, which is a chronic liver disease caused by progressive inflammation and scarring of the bile ducts inside and /or outside the liver. The inflammation impedes the flow of bile to the gut, which can ultimately lead to cirrhosis, liver failure and liver cancer. This inflammation is also associated with induction of inflammatory changes in the liver and in the bile ducts, and can also lead to cancer. Without wishing to be limited by a single hypothesis, the underlying cause of the inflammation is believed to be immune-mediated.

The present invention, in at least some embodiments, also relates to all subtypes of PSC including those involving small and large ducts as well as those involving the liver and the extra-hepatic ducts.

Currently, the only treatment for PSC is liver transplantation, which is clearly disadvantageous for many reasons. Thus, there is clearly an unmet need for a treatment for PSC that does not involve liver transplantation—a need which is met by the present invention in at least some embodiments.

Similarly, according to at least some embodiments, the present invention relates to PBC, which is an autoimmune disease of the liver, in which the small bile ducts (bile canaliculi) within the liver undergo progressive damage, leading to their destruction. As the ducts become more damaged, bile builds up in the liver (cholestasis), leading to scarring, fibrosis and cirrhosis. Again, the only treatment for PBC is liver transplantation, which is clearly disadvantageous for many reasons. Thus, there is clearly an unmet need for a treatment for PBC that does not involve liver transplantation—a need which is met by the methods described herein, in at least some embodiments.

According to at least some embodiments of the present invention, there are provided methods for treating PSC or PBC by administering an anti-CD3 immune molecule, such as an anti-CD3 antibody, orally or mucosally, for example and without limitation, via oral, pulmonary, buccal, nasal, intranasal, or sublingual administration. The PSC or PBC may optionally be caused by any factor or combinations of factors, and/or have any etiology, such as those described herein.

In at least some embodiments, there are provided pharmaceutical compositions for treatment of PSC or PBC suitable for oral or mucosal administration including an anti-CD3 antibody or fragment thereof (or any other suitable anti-CD3 immune molecule). Although the description centers around anti-CD3 antibodies, it is understood that the term “anti-CD3 antibody” includes antibodies, antigen-binding fragments thereof, or any other suitable anti-CD3 immune molecule. Also provided are anti-CD3 immune molecules for use in the preparation of a medicament for the treatment of PSC or PBC.

In some embodiments, the pharmaceutical composition is suitable for oral, pulmonary, buccal, nasal, intranasal, sublingual, rectal, or vaginal administration. In some embodiments, the anti-CD3 antibody is selected from the group consisting of a murine mAb (monoclonal antibody), a humanized mAb, a human mAb, and a chimeric mAb, which can bind to CD3. In some embodiments, the composition suitable for oral administration is in a form selected from a liquid oral dosage form and a solid oral dosage form, e. g., selected from the group consisting of tablets, capsules, caplets, powders, pellets, granules, powder in a sachet, enteric-coated tablets, enteric-coated beads, encapsulated powders, encapsulated pellets, encapsulated granules, and enteric-coated soft gel capsules. In some embodiments, the oral dosage form is a controlled release oral formulation.

In some embodiments, the pharmaceutical compositions further comprise excipients and/or carriers. In some embodiments, the pharmaceutical compositions further comprise additional active or inactive ingredients.

In an additional aspect, in at least some embodiments, the present invention provides methods of providing an anti-CD3 antibody to a subject for treatment of PSC or PBC. The methods for treatment of PSC or PBC can include administering to the subject an oral dosage form suitable to deliver a dosage of an anti-CD3 antibody via the gastrointestinal tract, which, upon oral administration, leads to amelioration of PSC or PBC and inflammation.

According to at least some embodiments, the present invention provides methods of providing an anti-CD3 antibody to a subject for treatment of PSC or PBC. The methods include administering to the subject an oral dosage form suitable to deliver a dosage of an anti-CD3 antibody via the gastrointestinal tract, which, without wishing to be limited by a single hypothesis, upon oral administration leads to stimulating the development of Tregs with resultant amelioration in PSC or PBC.

Alternatively, the methods for treatment of PSC or PBC may optionally include administering to the subject a mucosal dosage form suitable to deliver a dosage of an anti-CD3 antibody via a mucous membrane, which, upon mucosal administration and again without wishing to be limited by a single hypothesis, leads to stimulating the development of Tregs with resultant amelioration in PSC or PBC.

The present invention, in at least some embodiments, provides numerous advantages, in addition to its efficacy for treatment of PSC or PBC. Without wishing to be limited to a closed list, these advantages over known methods of treatment include the following. First, oral or mucosal administration is easier to accomplish and is generally preferred over parenteral administration (e.g., intravenous or by injection) by the majority of subjects, due to the lack of needles and needlesticks associated with chronic therapy, hence resulting in improved compliance by subjects. Second, oral or mucosal administration facilitates chronic administration of the antibody. Third, oral or mucosal administration generally can avoid or reduce the negative side effects and pain associated with parenteral administration, including injection site pain. Fourth, oral or mucosal administration can avoid the serious side effects associated with parenteral administration of antibody, including generalized immunosuppression and cytokine storm.

Other advantages include but are not limited to reduced costs, since highly trained personnel are not required for oral or mucosal administration, and fewer safety concerns for both subjects and medical staff that are using sharp needles. In some circumstances but without wishing to be limited by a closed list, orally or mucosally administered anti-CD3 antibodies result in reduced inflammation and/or autoimmune disease at a lower dosage than parenterally administered anti-CD3 antibodies and without the side effects of parenteral administration.

Moreover, oral or mucosal antibodies can be effective at multiple points in the disease cycle, for example when administered before development of the disease, during the ascending period of disease and when given at the peak of the disease, while parenterally administered antibodies are commonly believed to be effective only after onset of the disease (Chatenoud et al., J. Immunol. 158: 2947-54, 1997; Tran et al., Int. Immunol. 13: 1109-20, 2001).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION

The present invention, in at least some embodiments, provides methods of treating PSC or PBC via oral or mucosal administration of anti-CD3 antibodies and compositions suitable for oral or mucosal administration of anti-CD3 antibodies.

As described herein, PSC or PBC may be treated through oral or mucosal administration of an anti-CD3 immune molecule therapy. The usefulness of an oral formulation requires that the active agent be bioavailable. Bioavailability of orally administered drugs can be affected by a number of factors, such as drug absorption throughout the gastrointestinal tract, stability of the drug in the gastrointestinal tract, and the first-pass effect. Thus, effective oral delivery of an active agent requires that the active agent have sufficient stability during traversal of the stomach and intestinal lumen to reach and pass through the intestinal epithelium to the lamina propria. Many drugs, however, tend to degrade quickly in the intestinal tract or have poor absorption in the intestinal tract so that oral administration is not an effective method for administering the drug. Surprisingly, not only can anti-CD3 antibodies be administered orally, but oral administration is, in some aspects but without wishing to be limited by a closed list, superior to parenteral administration in terms of positive immune-modulatory activity for Tregs and in a practical way in terms of tolerability and ease of administration.

Within the immune system, a series of anatomically distinct compartments can be distinguished, each specially adapted to respond to pathogens present in a particular set of body tissues. One compartment, the peripheral compartment, comprises the peripheral lymph nodes and spleen; this compartment responds to antigens that enter tissues or spread into the blood. A second compartment, the mucosal immune system, is located near the mucosal surfaces where most pathogens invade. The mucosal immune system has evolved antigen-specific tolerance mechanisms to avoid a deleterious immune response to food antigens and beneficial commensal microorganisms that live in symbiosis with their host, while detecting and killing pathogenic organisms that enter through the gut. Generally speaking, the gut-associated lymphoid tissue (GALT) is different from other lymphoid tissue, in that stimulation of the GALT preferentially induces Tregs. Anti-CD3 immune molecules, e.g., anti-CD3 antibodies, are rapidly taken up by the GALT and induce CD4⁺CD25⁻LAP⁺ Treg. The cells in the GALT secrete mainly TGF-B and IL-10, and the chance and the frequency of stimulating Tregs is higher in the gut.

Immune responses induced within one compartment are largely confined to that particular compartment. Lymphocytes are restricted to particular compartments by their expression of homing receptors that are bound by ligands, known as addressins, which are specifically expressed within the tissues of that compartment. Interestingly, tolerance induced in the mucosal compartment also applies and transfers to the peripheral compartment. For example, the feeding of ovalbumin (a strong parenteral antigen) is followed by an extended period during which the injection of ovalbumin, even in the presence of adjuvant, elicits no antibody response in either the peripheral compartment or the mucosal compartment. In contrast, oral tolerance is a systemic tolerance; although oral tolerance is induced in the gut, peripheral tolerance also results.

Without wishing to be limited by a single hypothesis, orally administered anti-CD3 immune molecules are believed to stimulate the mucosal immune system. As noted above, the gut is a unique environment in which to induce tolerance. In comparison with parenterally administered antibodies, lower amounts of oral anti-CD3 antibodies are needed to induce tolerance and do so without stimulating general immune-suppression and other serious side effects; in addition, oral or mucosal antibodies can be effective at multiple points in the disease cycle, for example when administered before development of the disease, during the ascending period of disease and when given at the peak of the disease, while parenterally administered antibodies are effective only after onset of the disease.

Pharmaceutical Compositions

Pharmaceutical compositions suitable for oral administration are typically solid dosage forms (e.g., tablets) or liquid preparations (e.g., solutions, suspensions, emulsions, or elixirs).

Solid dosage forms are desirable for ease of determining and administering defined dosage of active ingredient, and ease of administration, particularly administration by the subject at home.

Liquid dosage forms also allow subjects to easily take the required dose of active ingredient; liquid preparations can be prepared as a drink, or to be administered, for example, by a naso-gastric tube.

Liquid oral pharmaceutical compositions generally require a suitable solvent or carrier system in which to dissolve or disperse the active agent, thus enabling the composition to be administered to a subject. A suitable solvent system is compatible with the active agent and non-toxic to the subject. Typically, liquid oral formulations use a water-based solvent.

The oral compositions can also optionally be formulated to reduce or avoid the degradation, decomposition, or deactivation of the active agent by the gastrointestinal system, e.g., by gastric fluid in the stomach. For example, the compositions can optionally be formulated to pass through the stomach unaltered and to dissolve in the intestines, i.e., as enteric coated compositions.

One of ordinary skill in the art would readily appreciate that the pharmaceutical compositions described herein can be prepared by applying known pharmaceutical manufacturing procedures as established through a long history of application for oral products. Such formulations can be administered to the subject with methods well-known in the pharmaceutical arts. Thus, the practice of the present methods will employ, unless otherwise indicated, conventional techniques of pharmaceutical sciences including pharmaceutical dosage form design, drug development, and pharmacology, as well as of organic chemistry, including polymer chemistry. Accordingly, these techniques are within the capabilities of one of ordinary skill in the art and are explained fully in the literature (See generally, for example, Remington: The Science and Practice of Pharmacy, Nineteenth Edition. Alfonso R. Gennaro (Ed.): Mack Publishing Co., Easton, Pa., (1995), hereinafter Remington, incorporated by reference herein in its entirety).

Anti-CD3 Immune Molecules

An anti-CD3 immune molecule may optionally comprise any anti-CD3 antibody. The anti-CD3 antibodies can be any antibodies specific for CD3. The term “antibody” as used herein refers to an immunoglobulin molecule or immunologically active portion thereof that is readily derived by means of known techniques of protein chemistry and recombinant DNA engineering, i.e., an antigen-binding portion. Non-limiting examples of immunologically active portions of immunoglobulin molecules include F(ab) and F(ab′)₂ fragments, which retain the ability to bind CD3. Such fragments can be obtained commercially or by using methods known in the art. For example, F(ab)₂ fragments can be generated by treating the antibody with an enzyme such as pepsin, a non-specific endopeptidase that normally produces one F(ab)₂ fragment and numerous small peptides of the Fc portion. The resulting F(ab)₂ fragment is composed of two disulfide-connected Fab units. The Fc fragment is extensively degraded and can be separated from the F(ab)₂ by dialysis, gel filtration or ion exchange chromatography. F(ab) fragments can be generated using papain, a non-specific thiol-endopeptidase that digests IgG molecules in the presence of a reducing agent, into three fragments of similar size: two Fab fragments and one Fc fragment. When Fc fragments are of interest, papain is the enzyme of choice because it yields a 50 kilodalton (kD) Fc fragment; to isolate the F (ab) fragments, the Fc fragments can be removed, e. g., by affinity purification using protein A or G. A number of kits are available commercially for generating F(ab) fragments, including the ImmunoPure IgG1 Fab and F(ab′)₂ Preparation Kit (Pierce Biotechnology, Rockford, Ill.). In addition, commercially available services for generating antigen-binding fragments can be used, e.g., Bio Express, West Lebanon, N.H.

The antibody may optionally be a polyclonal, monoclonal, recombinant, e.g., a chimeric, humanized, fully human, non-human, e.g., murine, or single chain antibody, that may optionally be deimmunized.

In some embodiments the antibody has effector function and can fix complement. In some embodiments, the antibody has reduced or no ability to bind an Fc receptor. For example, the anti-CD3 antibody can be an isotype or subtype, fragment or other mutant, which does not support binding to an Fc receptor, e. g., it has a mutagenized or deleted Fc receptor binding region. The antibody can be coupled to a toxin or imaging agent.

A number of anti-CD3 antibodies are known, including but not limited to OKT3 (muromonab/Orthoclone OKT3, Ortho Biotech, Raritan, NJ; U.S. Pat. No. 4,361,549); hOKT3Y1 (Herold et al, N.E.J.M. 346: 1692-8, 2002; HuM291 (Nuvion™, Protein Design Labs, Fremont, Calif.); gOKT3-5 (Alegre et al, J. Immunol. 148: 3461-8, 1992; 1F4 (Tanaka et al, J. Immunol. 142: 2791-5, 1989) ; G4.18 (Nicolls et al, Transplantation 55: 459-68, 1993) ; 145-2C11 (Davignon et al, J. Immunol. 141: 1848-54, 1988); and as described in Frenken et al, Transplantation 51: 881-7, 1991; U.S. Pat. Nos. 6,491, 916, 6,406,696, and 6,143,297; and/or U.S. Provisional Application No. 61/659,717, filed Jun. 14, 2012, owned in common with the present application and sharing at least one inventor). However any suitable anti-CD3 antibody may be used with the methods and compositions of the present invention.

Methods for making such antibodies are also known. A full-length CD3 protein or antigenic peptide fragment of CD3 can be used as an immunogen, or can be used to identify anti-CD3 antibodies made with other immunogens, e. g., cells, membrane preparations, and the like, e. g., E-rosette-positive purified normal human peripheral T cells, as described in U.S. Pat. No. 4,361,549 and 4,654,210. The anti-CD3 antibody can bind an epitope on any domain or region on CD3 for retaining functionality.

Chimeric antibodies contain portions of two different antibodies, typically of two different species. Generally, such antibodies contain human constant (C) regions and variable (V) regions from another species, e.g., murine V regions. For example, mouse/human chimeric antibodies have been reported that exhibit binding characteristics of the parental mouse antibody and effector functions associated with the human C region (e.g., Cabilly et al, U.S. Pat. No.4,816,567; Shoemaker et al, U.S. Pat. No. 4,978,745; Beavers et al., U.S. Pat. No. 4,975,369; and Boss et al., U.S. Pat. No. 4,816,397, all of which are incorporated by reference herein. Generally, these chimeric antibodies are constructed by preparing a genomic gene library from DNA extracted from pre-existing murine hybridomas (Nishimura et al. Cancer Research, 47: 999, 1987). The library is then screened for V-region genes from both heavy (H) and light (L) chains exhibiting the correct antibody fragment rearrangement patterns. Alternatively, cDNA libraries are prepared from RNA extracted from the hybridomas and screened, or the V regions are obtained by polymerase chain reaction. The cloned V-region genes are ligated into an expression vector containing cloned cassettes of the appropriate H or L chain human C region gene. The chimeric genes can then be expressed in a cell line of choice, e. g., Chinese hamster ovary cells. Such chimeric antibodies have been used in human therapy.

Humanized antibodies are known in the art. “Humanization” results in a less immunogenic antibody, with complete retention of the antigen-binding properties of the original molecule. In order to retain all antigen-binding properties of the original antibody, the structure of its combining-site has to be faithfully reproduced in the “humanized” version. This can potentially be achieved by transplanting the combining site of the nonhuman antibody onto a human framework, either (a) by grafting the entire nonhuman V domains onto human C regions to generate a chimeric antibody (Morrison et al., Proc. Natl. Acad. Sci., USA 81: 6801, 1984; Morrison and Oi, Adv. Immunol. 44: 65, 1988, which preserves the ligand-binding properties but also retains the immunogenicity of the nonhuman V domains); (b) by grafting only the nonhuman CDRs onto human framework and C regions with or without retention of critical framework residues (Jones et al. Nature, 321: 522, 1986; Verhoeyen et al., Science 239: 1539, 1988); or (c) by transplanting the entire nonhuman V domains (to preserve ligand-binding properties) but also “cloaking” (also termed “veneering”) them with a human-like surface through judicious replacement of exposed residues to reduce immunogenicity (Padlan, Molec. Immunol. 28: 489, 1991).

However, given use of the oral or mucosal delivery routes of the antibodies according to at least some embodiments of the present invention, such humanization or reduced immunogenicity may not be necessary.

The anti-CD3 antibody can also be a single chain antibody. A single-chain antibody (scFV) can be engineered (see, for example, Colcher et al., Ann. N.Y. Acad. Sci. 880: 263-80, 1999; and Reiter, Clin. Cancer Res. 2: 245-52, 1996). The single chain antibody can be dimerized or multimerized to generate multivalent antibodies having specificities for different epitopes of the same target CD3 protein. In some embodiments, the antibody is monovalent, e.g., as described in Abbs et al., Ther. Immunol. 1: 325-31, 1994, incorporated herein by reference.

The term “native antibodies and immunoglobulins” as used herein refer to heterotetrameric glycoproteins of about 150 kilodaltons, composed of two identical light (L) chains and two identical heavy (H) chains. Each L chain is linked to a H chain by one covalent disulfide bond, while the number of disulfide linkages varies between H chains of different Ig isotypes. Each H and L chain also has regularly spaced intrachain disulfide linkages. Each H chain has at one end a variable domain (VH) followed by a number of constant domains (CH). Each L chain has a variable domain at one end (VL) and one constant domain (CL) at its other end; the CL domain is aligned with the first CH domain, and the VL domain is aligned with the VH domain. Particular amino acid residues form an interface between the VH and VL domains (Chothia et al, J. Mol. Biol. 186: 651, 1985; Novotny and Haber, Proc Natl Acad Sci USA, 82: 4592, 1985; Chothia et al., Nature 342: 877, 1989).

The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4.

The VH and VL domains contain a binding domain (composed of CDRs) that interacts with and binds to an antigen. The C regions may mediate the binding of the Ig to host tissues or factors, including various cells of the immune system (e. g., effector cells) and the first component (Clq) of the classical complement system.

The term “Kabat numbering scheme” is a widely-adopted standard for numbering the amino residues of an Ab in a consistent manner, see, e.g., bioinf.org.uk/abs. It is based on sequence variability and is most commonly used to define the CDR sequence.

The terms “monoclonal antibody” (mAb) or “monoclonal antibody composition” as used herein refer to a preparation of antibody molecules of a single molecular composition. A mAb composition displays a single binding specificity and affinity for a particular epitope. The term “epitope” means a protein determinant capable of specific binding by an Ab. Epitopes usually consist of chemically active surface groupings of molecules such as amino acids or sugar side chains and usually have specific three-dimensional structural characteristics, as well as specific charge characteristics.

The phrases “an antibody recognizing an antigen” and “an antibody specific for an antigen” are used interchangeably herein with the term “an antibody which binds specifically to an antigen.”

The term “antigen-binding portion” of an Ab (or simply “antibody portion”), as used herein, refers to one or more fragments of an intact Ab that retain the ability to bind specifically to CD3 or an interaction thereof as described above. It has been shown that the antigen-binding function of an Ab can be performed by fragments of a full-length Ab. Examples of binding include (i) a Fab fragment, a monovalent fragment consisting of the VL, VH, CL and CH1 domains; (ii) a F(ab)′₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al, Nature 341: 544-6, 1989), which consists of a VH domain; (vi) an isolated CDR; and (vii) a nanobody, a H region containing a single V and two C domains. Furthermore, although the VL and VH domains of the Fv fragment are encoded by separate genes, they can be joined using recombinant methods by a synthetic linker that enables them to be made as a single protein chain in which the VL and VH regions pair to form monovalent molecules (known as single chain Fv (scFv); see e.g., Huston et al, Proc Natl Acad Sci USA 85: 5879-83, 1988). Such single chain antibodies are also intended to be encompassed within the term “antigen-binding portion” of an Ab. These Ab fragments are obtained using conventional techniques known to those with skill in the art, and the fragments are screened for utility in the same manner as are intact Abs.

The term “fragments” as used herein refers to sequences sharing at least 10% amino acids in length with the respective sequence of the intact or full length Ab, e.g., mAbs (native). These sequences can be used as long as they exhibit the same properties as the native sequence from which they derive. In some embodiments, a fragment can be at least 6 amino acids in length, and can be, for example, at least 8, at least 10, at least 14, at least 16, at least 17, at least 18, at least 19, at least 20 or at least 25 amino acids or greater than 25 amino acids from the full length protein from which the fragment was derived. In some embodiments, the term fragment encompasses at least 6, 10, 20, 50, 100, 250, 500 amino acids from the full length protein from which the fragment was derived. Exemplary fragments include C-terminal truncations, N-terminal truncations, or truncations of both C- and N-terminals (e.g., deletions of 1, 2, 3, 4, 5, 8, 10, 15, 20, 25, 40, 50, 75, 100 or more amino acids deleted from the N-termini, the C-termini, or both). Preferably these sequences share more than 80%, in particular more than 90% amino acids in length with the respective sequence of the intact or full length antibody, e.g., mAbs. In some embodiments, the term “fragments” as used herein, when used in reference to mAb fragments or antigen-binding portions or fragments, usually refers to a portion of at least 2, or at least about 5, or at least about 6, or at least about 8, or at least about 10 or more consecutive amino acids of the epitope binding region of an Ab. In some embodiments, a fragment includes at least 2, or at least about 5, or at least about 6, or at least about 8, or at least about 10 or more consecutive amino acids of the epitope binding region of an Ab having a sequence as described herein. In some embodiments, a fragment is a CDR region of at least 3 consecutive amino acids from any of the Ab sequences described herein. In some embodiments, a fragment is a CDR region selected from any and a combination of CDRs listed herein.

In some embodiments, the fragment is a functional fragment, where a “functional fragment” as used in the context of a “functional fragment of an antibody” refers to a fragment of the Ab that mediates the same effect as the full length Ab, e.g., specifically binds to the same antigen with the same or higher affinity compared to the full length Ab. In some embodiments, a functional fragment is a CDR region of at least 3 consecutive amino acids as described herein. In some embodiments, a functional fragment is a CDR region selected from any and a combination of CDRs, according to the CDR sequences provided herein.

In some embodiments, the mAb is bi-specific or multi-specific.

In the case of an Ab, e.g., mAb according to at least some embodiments of the present invention, useful fragments include, but are not limited to: a CDR region, especially a CDR3 region of the H or L chain; a VH or VL domain; a portion of an Ab chain or just its V region including two CDRs; and the like. In some embodiments, useful functional fragments include, but are not limited at least one or any combination of CDRs from the same Ab, as described herein.

Suitable Abs, e.g., mAb or fragments of the invention, are active, i.e., are immunologically functional Igs. The term “immunologically functional immunoglobulin fragment” as used herein refers to a polypeptide fragment that binds to CD3 and/or blocking one or more interactions of CD3 with another partner or partners, such as another protein for example. Such interaction may also optionally relate to steric hindrance of one or more interactions between CD3 and another partner or partners.

Optionally, suitable Abs, e.g., mAbs or isolated mAb fragments or antigen-binding portions or fragments thereof may be produced by a method comprising the steps of:

(a) producing a preparation of an antigen related to CD3, or a fragment, or a cell containing CD3 or a fragment, or a fusion protein thereof, of any species, e.g., human and/or vertebrate species;

(b) immunizing a rodent, e.g., a mouse, with the antigen, or a fragment, or a fusion protein thereof, or a cell containing said antigen;

(c) detecting specifically binding or blocking Abs in the serum of the mice;

(d) producing hybridomas between lymph node cells from the mice and myeloma cells to produce Abs; and

(e) screening hybridomas with an antigen-specific binding assay.

In some embodiments, an antigen used to produce Abs is human, or mouse, or from another mammalian species, or from another vertebrate species.

In some embodiments, the antigen used to produce Abs is a primary cell or cell line expressing endogenous or recombinant full length antigen or an antigenic fragment of the antigen, or a fusion protein of all or part of the antigen and another protein, or the antigen is part of a virus-like particle.

Optionally, the antigen used to produce Abs is expressed in a cell line syngeneic with mice of step (b), or the antigen used to produce Abs is fused to the Fc portion of an IgG.

Optionally, the antigen used to produce Abs is human or mouse antigen fused to the Fc portion of human IgG1.

Optionally, the antigen used to produce Abs is on the surface of cells.

As an alternative to steps b), c) and d), an antibody, or fragment thereof such as single chain Fv, can be obtained by selecting antibody sequences by phage display on the antigen of step a).

Optionally, the binding assay of step (e) is carried out by applying visualizing methods comprising enzyme-linked immunosorbent assay (ELISA), dot blot, immunoblot, RIA, immunoprecipitation, flow cytometry, fluorescence microscopy, electron microscopy, confocal microscopy, calorimetry, surface plasmon resonance, test of Ouchterlony, complement-mediated lysis of red blood cells, antibody-dependent cell cytotoxicity and the like. More preferably, the binding assay for a mAb is carried out by direct or capture ELISA or flow cytometry.

In particular, Abs can be purified, for example by protein A or G affinity chromatography or by protein L, anti-mouse IgG Ab-based affinity chromatography, ion exchange, ethanol or ammonium sulfate precipitation, and the like.

Methods for preparing an immunogen and immunizing an animal are well-known in the art (Kohler and Milstein, Nature 256: 495-7, 1975; Brown et al, J Immunol 127: 539-46, 1981; Brown et al, J Biol Chem 255: 4980-3, 1980; Yeh et al, Proc Natl Acad Sci USA 76: 2927-31, 1976; Yeh et al, Int J Cancer 29: 269-75, 1982; Kozbor et al, Immunol Today 4:72, 1983; Cole et al, Monoclonal Antibodies and Cancer Therapy, Alan R. Liss, Inc., pp. 77-96, 1985; U.S. Pat. No. 4,816,567; Clackson et al, Nature 352: 624-8 1991; Marks, et al J Mol Biol 222: 581-97, 1991).

According to some embodiments of the present invention, there are provided host cells comprising an expression vector containing a DNA segment encoding a signal peptide, consensus mouse H or L chain signal sequences, and a DNA segment encoding and expressing an anti-CD3 Ab as described herein, e.g., a mAb or isolated mAb fragments or antigen-binding portions or fragments thereof, as well as transgenic animals having a genome comprising said isolated DNA segment and/or the expression vector.

The terms “expression vector” and “recombinant expression vector” as used herein refer to a DNA molecule, for example a plasmid or modified virus, containing a desired and appropriate nucleic acid sequence necessary for the expression of the recombinant polypeptides in a host cell. As used herein, “operably linked” refers to a functional linkage of at least two sequences. Operably linked includes linkage between a promoter and a second sequence, for example a nucleic acid of the present invention, wherein the promoter sequence initiates and mediates transcription of the DNA sequence corresponding to the second sequence.

The regulatory regions necessary for transcription of the polypeptides can be provided by the expression vector. The precise nature of the regulatory regions needed for gene expression may vary among vectors and host cells. Generally, a promoter is required which is capable of binding RNA polymerase and promoting the transcription of an operably-associated nucleic acid sequence. Regulatory regions may include those 5′ non-coding sequences involved with initiation of transcription and translation, such as the TATA box, cap sequence, CAAT sequence, and the like. The non-coding region 3′ to the coding sequence may contain transcriptional termination regulatory sequences, such as terminators and polyadenylation sites. A translation initiation codon (ATG) may also be provided. The vector design for mammalian expression cells may contain leader sequences, one common for the H chain and one common to the L chain, for enabling high levels of mAb expression and secretion. The N-terminal peptide signal sequence is initially synthesized on the ribosome which is recognized by the signal recognition particle (SRP) that stalls mRNA translation while the ribosome is docked via the signal sequence to the SEC61 translocon at which point the SRP is dissociated and mRNA translation resumes with the feeding of the polypeptide into the ER. The signal sequence thus can play a crucial role in the synthesis of mAbs.

In order to clone the nucleic acid sequences into the cloning site of a vector, linkers or adapters providing the appropriate compatible restriction sites are added during synthesis of the nucleic acids. For example, a desired restriction enzyme site can be introduced into a fragment of DNA by amplification of the DNA by use of PCR with primers containing the desired restriction enzyme site.

An alternative method to PCR is the use of a synthetic gene. The method allows production of an artificial gene that comprises an optimized sequence of nucleotides to be expressed in host cells of a desired species (e.g., CHO cells or E. coli). Redesigning a gene offers a means to improve gene expression in many cases. Rewriting the open reading frame (ORF) is possible because of the redundancy of the genetic code. Thus it is possible to change up to about one-third of the nucleotides in an ORF and still produce the same protein. For a typical protein sequence of 300 amino acids, there are over 10150 codon combinations that will encode an identical protein. Using optimization methods such as replacing rarely used codons with more common codons can result in dramatic effects. Further optimizations such as removing RNA secondary structures can also be included. Computer programs are available to perform these and other simultaneous optimizations. A well-optimized gene can dramatically improve protein expression. Because of the large number of nucleotide changes made to the original DNA sequence, the only practical way to create the newly designed gene is to use gene synthesis.

An expression construct comprising a polypeptide sequence operably associated with regulatory regions can be directly introduced into appropriate host cells for expression and production of polypeptide per se or as a recombinant fusion protein. The expression vectors that may be used include but are not limited to plasmids, cosmids, phage, phagemids or modified viruses. Typically, such expression vectors comprise a functional origin of replication for propagation of the vector in an appropriate host cell, one or more restriction endonuclease sites for insertion of the desired gene sequence, and one or more selection markers.

The recombinant polynucleotide construct comprising the expression vector and a polypeptide according to the invention should be transferred into a host cell where it can replicate (e.g., a bacterial cell), and then be transfected and expressed in an appropriate prokaryotic or eukaryotic host cell. This can be accomplished by methods known in the art. The expression vector is used with a compatible prokaryotic or eukaryotic host cell which may be derived from bacteria, yeast, insects, mammals and humans.

The term “mutant” or “variant” as used herein in reference to an amino acid, DNA or RNA sequence means that such a sequence differs from, but has sequence identity with, the wild-type or disclosed sequence. The degree of sequence identity between the wild-type or disclosed sequence and the mutant sequence is preferably greater than about 50%, and in many cases is about 60%, 70%, 80%, 90%, 95, 98% or more.

The amino acid residues referred to herein encompass the natural coded amino acids represented by either one-letter or three-letter codes according to conventions well known in the art. In chemical synthesis, amino acid derivatives and D isomers can also be used. In chemical synthesis, sequential, divergent and convergent synthetic approaches to the peptide sequence may be used.

The terms “protein” and “polypeptide” are used interchangeably herein to refer to amino acids joined to each other by peptide bonds or modified peptide bonds, i.e., peptide isosteres, and may contain modified amino acids other than the 20 gene-encoded amino acids. The polypeptides may be modified by either natural processes, such as post-translational processing, or by chemical modification techniques which are well known in the art. Modifications, pre- or post-translational, can occur anywhere in the polypeptide, including the peptide backbone, the amino acid side-chains and the amino or carboxyl termini It will be appreciated that the same type of modification may be present in the same or varying degrees at several sites in a given polypeptide. Also a given polypeptide may have many types of modifications.

Modifications of polypeptides and amino acids include acetylation; acylation; ADP-ribosylation; amidation; covalent attachment of non-peptide molecules such as flavin, a heme moiety, a nucleotide or nucleotide derivative, a lipid or lipid derivative or phosphytidylinositol; cross-linking cyclization; disulfide bond formation; demethylation; formation of covalent cross-links; formation of cysteine; formation of pyroglutamate; formylation; gamma-carboxylation; glycosylation; GPI anchor formation; hydroxylation; iodination; methylation; myristolyation; oxidation; pegylation; proteolytic processing; phosphorylation; prenylation; racemization; selenoylation; sulfation; and transfer-RNA mediated addition of amino acids to protein such as arginylation (see, e.g., Creighton T E, Proteins-Structure and Molecular Properties 2nd Ed., W. H. Freeman and Company, New York, 1993; Posttranslational Covalent Modification of Proteins, B. C. Johnson, Ed., Academic Press, New York, pp. 1-12, 1983).

As used herein, “heterologous” refers to two biological components that are not found together in nature. The components may be proteins or fragments thereof, host cells, genes or control sequences such as promoters. Although the heterologous components are not found together in nature, they can function together, such as when a promoter heterologous to a gene is operably linked to the gene.

The terms “polynucleotide”, “nucleic acid sequence” and “nucleic acid” are used interchangeably herein to refer to polymeric forms of nucleotides of any length, either ribonucleotides or deoxynucleotides, including but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. Further included are mRNA or cDNA that comprise intronic sequences (see, e.g., Niwa et al, Cell 99: 691-702, 1999). The backbone of the polynucleotide can comprise sugars and phosphate groups (as typically be found in RNA or DNA), or modified or substituted sugar or phosphate groups. Alternatively, the backbone of the polynucleotide can comprise a polymer of synthetic subunits such as phosphoramidites and thus can be an oligodeoxynucleoside phosphoramidate or a mixed phosphoramidate-phosphodiester oligomer (see, e.g., Peyrottes et al, Nucl Acids Res 24: 1841-8, 1996; Chaturvedi et al, Nucl Acids Res 24: 2318-23, 1996). Polynucleotides may comprise modified nucleotides, such as methylated nucleotides and nucleotide analogs, uracil, other sugars, and linking groups such as fluororibose and thioate, and nucleotide branches. The sequence of nucleotides may be interrupted by non-nucleotide components. A polynucleotide may be further modified after polymerization, such as by conjugation with a labeling component, capping, substitution of one or more of naturally occurring nucleotides with an analog, and introduction of means for attaching the polynucleotide to proteins, metal ions, labeling components, other polynucleotides, or a solid support.

The terms “coding sequence of” and “coding region of”, in reference to a particular polypeptide or protein, are used interchangeably herein to refer to a nucleic acid sequence which is transcribed and translated into the particular polypeptide or protein when placed under the control of appropriate regulatory sequences.

The term “polynucleotide sequence encoding a polypeptide” encompasses a polynucleotide which includes only coding sequence for the polypeptide, as well as a polynucleotide which includes additional coding and/or non-coding sequence. Examples of additional coding sequences include leader or secretory sequences. Examples of non-coding sequences or regulatory sequences such as promoters, transcription enhancers, etc., are well known in the art.

The term “identity”, as used herein and as known in the art, is a relationship between two or more polypeptide sequences or two or more polynucleotide sequences, as determined by comparing the sequences. The term “identity” also means the degree of sequence relatedness between polypeptide or polynucleotide sequences, as the case may be, as determined by the match between strings of such sequences. “Identity” and “similarity” can be readily calculated by known methods (described, e.g., in, Computational Molecular Biology, Lesk A M, ed., Oxford University Press, New York, 1988; Biocomputing: Informatics and Genome Projects, Smith D W, ed., Academic Press, New York, 1993; Computer Analysis of Sequence Data, Part I, Griffin AM and Griffin HG eds., Humana Press, New Jersey, 1994; Sequence Analysis in Molecular Biology, von Heinje G, Academic Press, 1987; Sequence Analysis Primer, Gribskov M and Devereux J eds., M Stockton Press, New York, 1991; Carillo H and Lipman D, SIAM J. Applied Math 1988, 48: 1073).

Preferred methods to determine identity are designed to give the largest match between the tested sequences. Methods to determine identity and similarity are codified in publicly available computer programs. Preferred program methods to determine identity and similarity between two sequences include, but are not limited to, the GCG program package (Devereux et al, Nucl Acids Res 24: 2318-23, 1984), BLASTP, BLASTN, and FASTA (Atschul et al, J Molec Biol 215: 403-10, 1990). The BLAST X program is publicly available from NCBI and other sources (BLAST Manual, Altschul et al, NCBI NLM NIH Bethesda, MD 20894; Altschul et al, J Molec Biol 215: 403-10, 1990). As an illustration, by a polynucleotide having a nucleotide sequence having at least, for example, 95% “identity” to a reference nucleotide sequence, it is intended that the nucleotide sequence of the tested polynucleotide is identical to the reference sequence over its full length. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

The phrase “substantially identical” in the context of two nucleic acids or polypeptides, refers to two or more sequences that have at least 50%, 60%, 70%, 80%, and in some aspects 90-95% nucleotide or amino acid residue identity, when compared and aligned for maximum correspondence, as measured using one of the known sequence comparison algorithms or by visual inspection. In some embodiments, the sequences are substantially identical over the entire length of the coding regions.

A “substantially identical” amino acid sequence is one that differs from a reference sequence by one or more conservative or non-conservative substitutions, deletions or insertions, provided that the polypeptide generally retains its functional and/or immunogenic and/or antibody-binding properties. A conservative amino acid substitution, for example, substitutes one amino acid residue for another of the same class (e.g., substitution of a hydrophobic residue for another, such as isoleucine, valine, leucine, or methionine,; or substitution of a polar residue for another, such as substitution of arginine for lysine, glutamic acid for aspartic acid, or glutamine for asparagine).

The term “oligonucleotide” refers to a single-stranded polydeoxynucleotide or two complementary polydeoxynucleotide strands which may be chemically synthesized. Synthetic oligonucleotides generally lack 5′-phosphate and thus will not ligate to another oligonucleotide without adding a phosphate with an ATP in the presence of a kinase.

The term “primer” as used herein refers to an oligonucleotide which is capable of acting as a point of initiation of nucleic acid synthesis when placed under conditions in which synthesis of a primer extension product which is complementary to a nucleic acid strand is induced, i.e., in the presence of nucleotides and an inducing agent such as DNA polymerase and at a suitable temperature and pH. Primers may be obtained from a biological source, as in a purified restriction digest of genomic DNA, or produced synthetically. The primers are preferably single stranded for maximum efficiency in amplification, but may alternatively be double-stranded. If double-stranded, the primer is first treated to separate its strands before being used to prepare amplification products. Preferably, the primers are oligodeoxyribonucleotides but must be sufficiently long to prime the synthesis of extension products in the presence of the inducing agent. The exact lengths of the primers will depend on many factors, including temperature, source of primer and use of the method. The primers typically contain 10 or more nucleotides.

Synthetic oligonucleotide primers may be prepared using any suitable method, such as, for example, the phosphotriester and phosphodiester methods (Narang et al, Meth. Enzymol. 68: 90, 1979; Brown et al, Meth. Enzymol. 68: 109, 1979) or automated embodiments thereof. In one such automated embodiment, diethylphosphoramidites are used as starting materials and may be synthesized (as described by Beaucauge et al, Tetrahedron Let. 22: 1859-62, 1981). One method for synthesizing oligonucleotides on a modified solid support is described in U.S. Pat. No. 4,458,066 which is incorporated herein by reference.

The term “digestion” in reference to a nucleic acid, in particular a DNA molecule, refers to catalytic cleavage of the DNA with a restriction enzyme that acts only at certain sequences in the DNA. The various restriction enzymes used herein are commercially available and their reaction conditions, cofactors and other requirements may be readily ascertained by the ordinarily skilled artisan. After digestion, gel electrophoresis may be performed to isolate the desired fragment, the latter of which is also referred to as a “restriction fragment”.

As used herein, the term “isolated” means that the material is removed from its original environment. The original environment may be a natural environment if the material is naturally occurring, for example in a bacterial cell wall, or the original environment may be an artificial environment, if the material is artificial or engineered. For example, a naturally occurring polynucleotide or polypeptide present in a living organism, when separated from some or all of the coexisting materials in the natural system, is isolated. Similarly, a recombinantly engineered polynucleotide or the corresponding expressed polypeptide, are referred to as isolated, when separated from a vector or expression system respectively containing the recombinant polynucleotide or expressed polypeptide.

As used herein, the term “purified” does not require absolute purity; rather, it is intended as a relative definition. The purified nucleic acid sequences of the invention have been purified from other sequences, such as the remainder of genomic DNA or from other sequences in a library or other environment by at least one order of magnitude, typically two or three orders, and more typically four or five orders of magnitude, to a sufficient degree that enables further manipulation of the specific DNA sequence.

As used herein, the term “recombinant”, in reference to a nucleic acid, means that the nucleic acid is adjacent to a “backbone” nucleic acid to which it is not adjacent in its natural cellular or viral environment. Backbone molecules according to the invention include nucleic acids such as expression vectors, self-replicating nucleic acids, viruses, integrating nucleic acids, and other vectors or nucleic acids used to maintain or manipulate a nucleic acid insert of interest.

As used herein, the term “recombinant”, in reference to polypeptides or proteins, means polypeptides or proteins produced by recombinant DNA techniques, i.e., produced from cells transformed by a DNA construct encoding the desired polypeptide or protein.

As used herein, “host cell” refers to a cell that has been transfected or transformed or is capable of transfection or transformation by an exogenous polynucleotide sequence, either in the form of a recombinant vector or other transfer DNA, and includes the progeny of the original cell which has been transfected or transformed.

As used herein, the term “control sequence” refers to a nucleic acid having a base sequence which is recognized by the host organism to effect the expression of encoded sequences to which they are ligated. The nature of such control sequences differs depending upon the host organism; in prokaryotes, such control sequences generally include a promoter, ribosomal binding site, terminators, and in some cases operators; in eukaryotes, generally such control sequences include promoters, terminators and in some instances, enhancers. The term control sequence is intended to include at a minimum, all components whose presence is necessary for expression, and may also include additional components whose presence is advantageous, for example, leader sequences.

As used herein, the term “operably linked” refers to sequences joined or ligated to function in their intended manner. For example, a control sequence is operably linked to coding sequence by ligation in such a way that expression of the coding sequence is achieved under conditions compatible with the control sequence and host cell. For example, a promoter sequence is “operably linked to” a coding sequence when RNA polymerase which initiates transcription at the promoter will transcribe the coding sequence into mRNA.

As used herein, the term “synthetic” in reference to polypeptides or protein sequences, means those that are those prepared by chemical synthesis. Sequential, divergent and convergent synthetic approaches may be used in chemical synthesis.

Pharmaceutical Compositions with Anti-CD3 Antibodies

The anti-CD3 antibodies described herein can be incorporated into a pharmaceutical composition suitable for oral or mucosal administration, e.g., by ingestion, inhalation, or absorption, e.g., via oral, nasal, intranasal, pulmonary, buccal, sublingual, rectal, or vaginal administration. Such compositions can include an inert diluent or an edible carrier. For the purpose of oral therapeutic administration, the active compound (e.g., an anti-CD3 antibody) can be prepared with excipients and used in solid or liquid (including gel) form. Oral anti-CD3 antibody compositions can also be prepared using an excipient. Pharmaceutically compatible binding agents can be included as part of the composition. Oral dosage forms comprising anti-CD3 antibody are provided, wherein the dosage forms, upon oral administration, provide a therapeutically effective mucosal level of anti-CD3 antibody to a subject. Also provided are mucosal dosage forms comprising anti-CD3 antibody wherein the dosage forms, upon mucosal administration, provide a therapeutically effective mucosal level of anti-CD3 antibody to a subject. For the purpose of mucosal therapeutic administration, the active compound (e.g., anti-CD3 antibody) can be incorporated with excipients or carriers suitable for administration by inhalation or absorption, e.g., via nasal sprays or drops, or rectal or vaginal suppositories.

Solid oral dosage forms include, but are not limited to, tablets (e.g. chewable), capsules, caplets, powders, pellets, granules, powder in a sachet, enteric coated tablets, enteric coated beads, and enteric-coated soft gel capsules. Also included are multi-layered tablets, wherein different layers can contain different drugs. Solid dosage forms also include powders, pellets and granules that are encapsulated. The powders, pellets, and granules can be coated, e.g., with a suitable polymer or a conventional coating material to achieve, for example, greater stability in the stomach or gastrointestinal tract, or to achieve a desired rate of release. In addition, a capsule comprising the powder, pellets or granules can be further coated. A tablet or caplet can be scored to facilitate division for ease in adjusting dosage as needed.

The dosage forms of the present invention can be unit dosage forms wherein the dosage form is intended to deliver one therapeutic dose per administration, e.g., one tablet is equal to one dose. Such dosage forms can be prepared by methods of pharmacy well known to those skilled in the art (see Remington's Pharmaceutical Sciences, 18th ed., Mack Publishing, Easton Pa., 1990).

Typical oral dosage forms can be prepared by combining the active ingredients in an intimate admixture with at least one excipient according to conventional pharmaceutical compounding techniques. Excipients can take a wide variety of forms depending on the form of preparation desired for administration. For example, excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents. Examples of excipients suitable for use in oral liquid dosage forms include, but are not limited to, water, glycols, oils, alcohols, flavoring agents, preservatives, and coloring agents.

Tablets and capsules represent convenient pharmaceutical compositions and oral dosage forms, in which case solid excipients are employed. If desired, tablets can be coated by standard aqueous or non-aqueous techniques. Such dosage forms can be prepared by any of the methods of pharmacy. In general, pharmaceutical compositions and dosage forms are prepared by uniformly admixing the active ingredients with liquid carriers, finely divided solid carriers, or both, and then shaping the product into the desired presentation if necessary.

As one example, a tablet can be prepared by compression or by molding. Compressed tablets can be prepared, e.g., by compressing, in a suitable machine, the active ingredients (anti-CD3 antibody) in a free-flowing form such as powder or granules, optionally mixed with an excipient. Molded tablets can be made, e.g., by molding, in a suitable machine, a mixture of the powdered anti-CD3 antibody compound moistened, e.g., with an inert liquid diluent.

Excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gum tragacanth or gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidinones, methyl cellulose, pre-gelatinized starch, hydroxypropyl methyl cellulose, (e. g., Nos. 2208, 2906, 2910), microcrystalline cellulose, and mixtures thereof.

Suitable forms of microcrystalline cellulose include, but are not limited to, the materials sold as AVICEL PH-101, AVICEO PH-103 AVICEL RC-581, AVICEO PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEO RC-581. Suitable anhydrous or low moisture excipients or additives include AVICEL PH-103 and Starch 1500.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions and dosage forms of the invention is typically present in from about 50 to about 99 weight-percent of the pharmaceutical composition or dosage form.

Disintegrants can be used in the pharmaceutical compositions and oral or mucosal dosage forms of the invention to provide tablets that disintegrate when exposed to an aqueous environment. Tablets containing too much disintegrant might disintegrate during storage, while those containing too little might not disintegrate at a desired rate or under desired conditions.

Thus, a sufficient amount of disintegrant that is neither too much nor too little to detrimentally alter the release of the active ingredients should be used to form the pharmaceutical compositions and solid oral dosage forms described herein. The amount of disintegrant used varies based upon the type of formulation, and is readily discernible to those of ordinary skill in the art. Typically, pharmaceutical compositions and dosage forms comprise from about 0.5 to about 15 weight-percent of disintegrant, preferably from about 1 to about 5 weight-percent of disintegrant.

Disintegrants that can be used in pharmaceutical compositions and oral or mucosal dosage forms of the invention include, but are not limited to, agar-agar, alginic acid, calcium carbonate, Primogel, microcrystalline cellulose, croscarmellose sodium, crospovidone, polacrilin potassium, sodium starch glycolat corn, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate or Sterotes, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSILe 200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight-percent of the pharmaceutical compositions or dosage forms into which they are incorporated. A glidant such as colloidal silicon dioxide can also be used.

The pharmaceutical compositions and oral or mucosal dosage forms can further comprise one or more compounds that reduce the rate by which an active ingredient will decompose. Thus the oral dosage forms described herein can be processed into an immediate release or a sustained release dosage form Immediate release dosage forms may release the anti-CD3 antibody in a fairly short time, e.g., within a few minutes to a few hours. Sustained release dosage forms may release the anti-CD3 antibody over a period of several hours, e.g., up to 24 hours or longer, if desired. In either case, delivery can be controlled to be substantially at a certain predetermined rate over the period of delivery. In some embodiments, the solid oral dosage forms can be coated with a polymeric or other known coating material(s) to achieve, e.g., greater stability on the shelf or in the gastrointestinal tract especially for traversing the stomach's acidic pH, or to achieve control over drug release. Such coating techniques and materials used therein are well known in the art. Such compounds, which are referred to herein as “stabilizers”, include, but are not limited to, antioxidants such as ascorbic acid and salt buffers. For example, cellulose acetate phthalate, polyvinyl acetate phthalate, hydroxypropylmethyl cellulose phthalate, methacrylic acid-methacrylic acid ester copolymers, cellulose acetate trimellitate, carboxymethylethyl cellulose, and hydroxypropylmethyl cellulose acetate succinate, among others, can be used to achieve enteric coating. Mixtures of waxes, shellac, zein, ethyl cellulose, acrylic resins, cellulose acetate, silicone elastomers can be used to achieve sustained release coating. See, for example, Remington, supra, Chapter 93, for other types of coatings, techniques and equipment.

Liquids for oral or mucosal administration represent another convenient dosage form, in which case a solvent can be employed. In some embodiments, the solvent is a buffered liquid such as phosphate buffered saline (PBS). Liquid oral dosage forms can be prepared by combining the active ingredient in a suitable solvent to form a solution, suspension, syrup, emulsion, or elixir of the active ingredient in the liquid. The solutions, suspensions, syrups, emulsions and elixirs may optionally comprise other additives including, but not limited to, glycerin, sorbitol, propylene glycol, sugars or other sweeteners, flavoring agents, and stabilizers. Flavoring agents can include, but are not limited to peppermint, methyl salicylate, or orange flavoring.

Sweeteners can include sugars, aspartame, acesulfame-K, saccharin, sodium cyclamate and xylitol.

In order to reduce the degree of inactivation of orally administered anti-CD3 antibody in the stomach of the treated subject due to acidic pH, an antacid can be administered before simultaneously with the immunoglobulin, which neutralizes the otherwise acidic character of the gut. Thus in some embodiments, the anti-CD3 antibody is administered orally after or with an antacid, e.g., aluminum hydroxide or magnesium hydroxide such as MAALOX antacid or MYLANTA antacid, or an H2 blocker, such as cimetidine or ranitidine, or proton pump inhibitor such as a member of the benzimidazole family, such as omeprazole. One of skill in the art will appreciate that the dose of antacid administered in conjunction with an anti-CD3 antibody depends on the particular antacid used. When the antacid is MYLANTA antacid in liquid form, between 15 ml and 30 ml can be administered, e.g., about 15 ml. When the cimetidine H2 blocker is used, between about 400 and 800 mg per day can be used. When the proton pump inhibitor is used, between about 20 and 40 mg per day can be used.

Another method for reducing the degree of inactivation of orally administered anti-CD3 antibody in the stomach of the treated subject is to formulate the anti-CD3 in a suitable buffer of elevated pH with high buffering capacity.

The kits described herein can include an oral anti-CD3 antibody composition as an already prepared liquid oral dosage form ready for administration or, alternatively, can include an anti-CD3 antibody composition as a solid pharmaceutical composition that can be reconstituted with a solvent to provide a liquid oral dosage form. When the kit includes an anti-CD3 antibody composition as a solid pharmaceutical composition that can be reconstituted with a solvent to provide a liquid dosage form (e.g., for oral or nasal administration), the kit may optionally include a reconstituting solvent. In this case, the constituting or reconstituting solvent is combined with the active ingredient to provide a liquid oral dosage form of the active ingredient. Typically, the active ingredient is soluble in the solvent and forms a solution. The solvent can be, e.g., water, a non-aqueous liquid, or a combination of a non-aqueous component and an aqueous component. Suitable non-aqueous components include, but are not limited to oils, alcohols such as ethanol; glycerin, and glycols such as polyethylene glycol and propylene glycol. In some embodiments, the solvent is PBS.

For administration by inhalation, the mucosal anti-CD3 antibody compounds can be delivered in the form of an aerosol spray from pressured container or dispenser which contains a suitable propellant, e.g., a gas such as carbon dioxide, or a nebulizer. Such methods include those described in U.S. Pat. No. 6,468,798.

The anti-CD3 antibody compounds can also be prepared in the form of suppositories (e.g., with conventional suppository bases such as cocoa butter and other glycerides) or retention enemas for rectal or vaginal delivery, or for sprays for nasal or pulmonary delivery.

In one embodiment, the oral or mucosal anti-CD3 antibody compositions are prepared with carriers that will protect the anti-CD3 antibody against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522, 811.

Dosage, toxicity and therapeutic efficacy of such anti-CD3 antibody compositions can be determined by standard pharmaceutical procedures in cell cultures (e.g., of cells taken from an animal after mucosal administration of an anti-CD3 antibody) or experimental animals, e.g., for determining the LD₅₀ (the dose lethal to 50% of the study group) and the ED₅₀ (the dose therapeutically effective in 50% of the study group). The dose ratio between toxic and therapeutic effects is the therapeutic index, which can be expressed as the ratio LD₅₀/ED₅₀. Compositions which exhibit high therapeutic indices are preferred.

The data obtained from ex-vivo cell cultures (e.g., cells taken from an animal after mucosal administration of an anti-CD3 antibody) and animal studies can be used in formulating a range of dosage levels for use in humans. The dosage of anti-CD3 antibody compositions lies preferably within a range of mucosally available concentrations that include the ED₅₀ with little or no toxicity. The dosage may vary within this range depending upon the dosage form employed and the route of administration utilized. For any oral or mucosal anti-CD3 antibody compositions used in the methods described herein, the therapeutically effective dose can be estimated initially from assays of cell cultures (e.g., cells taken from an animal after mucosal administration of an anti-CD3 antibody). A dose also may be formulated in animal studies based on efficacy in suitable animal models. Such information can be used to more accurately determine useful doses in humans on the basis of differences in body mass.

As defined herein, a therapeutically effective amount of an anti-CD3 antibody (i.e., an effective dosage) depends on the antibody selected, the mode of delivery, and the condition to be treated. For instance, single dose amounts in the range of approximately 1 μg/kg to 1000 μg/kg may be administered; in some embodiments, about 5, 10, 50, 100, or 500 μg/kg may be administered. The anti-CD3 antibody compositions can be administered from one or more times per day to one or more times per week, including for example once every day. The oral or mucosal anti-CD3 antibody compositions can be administered, e.g., for about 10 days or longer. The skilled artisan will appreciate that certain factors may influence the dosage and timing required to effectively treat a subject, including but not limited to the severity of the disease or disorder, previous treatments, the general health and/or age of the subject, other diseases present, and persistence of the therapeutic effect.

Moreover, treatment of a subject with a therapeutically effective amount of the compounds may include a series of treatments.

The pharmaceutical compositions can be included in a container, pack, or dispenser together with instructions for administration.

Methods of Treatment

According to various embodiments of the present invention, the oral and mucosal anti-CD3 antibody compositions described herein can be administered to a subject to treat (which as described previously also includes preventing progression and/or delaying development of) PSC or PBC.

In some embodiments, the methods include administering an oral or mucosal anti-CD3 composition sufficient to produce an improvement in one or more clinical markers of PSC or PBC; for example, reduction or amelioration, or at least a reduction or absence of progression, of cirrhosis and/or fibrosis of the liver.

In some embodiments, the methods include screening the subject for clinical evidence of volume overload, uncontrolled hypertension, or uncompensated heart failure. In some embodiments, the methods include not administering the oral or mucosal anti-CD3 antibodies to subjects who have evidence of any of, volume overload, uncontrolled hypertension, or uncompensated heart failure. In some embodiments, the methods involve evaluating the subject's pulmonary function, and not administering the anti-CD3 antibodies to subjects who do not have a clear chest X-ray. In some embodiments, the methods include monitoring CD3⁺ T-cell clearance and/or plasma levels of anti-CD3 antibody, and adjusting the dosage of the oral or mucosal anti-CD3 compositions accordingly.

In some embodiments, the oral or mucosal anti-CD3 antibody compositions are administered concurrently with one or more second therapeutic modalities as described herein.

In some embodiments, the above treatment method may also optionally encompass monitoring liver function of the subject, before, during and/or after treatment. Liver function may optionally be assessed according to any known assay or test, including but not limited to a blood test (including but not limited to a test to assay one or more of alanine aminotransferase (ALT), aspartate aminotransferase (AST) or gamma-glutamyl transpeptidase (GGT) and/or any ratios thereof) and/or a liver biopsy (for example optionally as a needle biopsy).

In some embodiments the subject optionally does not have an autoimmune disease and/or optionally does not have diabetes.

### EXAMPLES

Some embodiments of the present invention are further described in the following example, which does not limit the scope of the invention described in the claims.

### Example 1

PSC Has No Suitable Animal Model for Pre-Clinical Testing Oral Anti-CD3 Immunotherapy Prior to Regulatory Approval for Clinical Trials

As an Orphan Disease, PSC is one of the more common chronic cholestatic liver diseases for which there is no approved treatment (Lee, Y. M. & Kaplan, M. M. Primary sclerosing cholangitis. New England Journal of Medicine 332, 924-933 (1995)). Although there is no agreement on etiology, evidence points to the immune system playing a role in, or being altered as a consequence of, PSC. Patients have a wide array of autoantibodies, indicating altered immune regulation. Among autoantibodies in PSC patients as well as in other autoimmune diseases are anti-yeast (ASCA), anti-neutrophil cytoplasm (ANCA), anti-smooth-muscle (ASMA), anti-nuclear (ANA), anti-endothelial cell (AECA), anti-cardiolipin, rheumatoid factor and others. CEP-hTMS-related epitopes have been proposed as a trigger for UC-associated PSC (DAS, K. M. Immunopathogenesis of primary sclerosing cholangitis: possible role of a shared colonic and biliary epithelial antigen. Journal of Gastroenterology and Hepatology 19, S290-S294 (2004)). Evidence for T-cell-mediated autoimmunity was found with respect to the predominant TcR V133 gene usage in PSC liver, indicating a possible autoantigen driving specific T cells to liver (Brommé, U., Grunewald, J., Scheynius, A., Olerup, O. & Hultcrantz, R. Preferential Vβ3 usage by hepatic T lymphocytes in patients with primary sclerosing cholangitis. Journal of hepatology 26, 527-534, doi:10.1016/s0168-8278(97)80417-5 (1997)). A genome-wide study found that PSC is associated with SNPs in genes encoding HLA-B with odds ratio=4.8 (Karlsen, T. H. et al. Genome-Wide Association Analysis in Primary Sclerosing Cholangitis. Gastroenterology 138, 1102-1111, doi:10.1053/j.gastro.2009.11.046 (2010)), suggesting an immunogenetic predisposition. Although the immune system seems to play a role in the pathogenesis of the disease, surprisingly, currently used immune modulatory agents failed to induce remission of the disease.

When evaluating a preclinical (animal) study of oral anti-CD3 in a PSC model from the perspectives of efficacy and safety, the key question is whether or not there is a satisfactory animal model for PSC in which to conduct such an evaluation. If so, a preclinical study can be beneficial as a prelude to a clinical trial. If not, then a preclinical study would not be useful, and the program should proceed directly to the clinic. Although various (19) animal models of PSC have been studied, no single model has been established exhibiting even most attributes of PSC disease. A complete picture of disease needs different models to study particular PSC pathogenetic steps. However, multiple models, each mimicking parts of pathogenesis, will not create a complete picture of efficacy for predicting clinical studies. Thus, as described in greater detail below, no existing animal model will be useful for a preclinical study of oral anti-CD3 immunotherapy in PSC.

Evaluations

Published reports of 19 animal models for PSC were evaluated. In order to study the effects of oral anti-CD3 immunotherapy on PSC, it is necessary to employ an animal model with the following attributes in order to be as relevant as possible to clinical PSC:

1. The model should have as many as possible of the following biological features, as highlighted by papers that reviewed the field of PSC (LaRusso, N. F. et al. Primary sclerosing cholangitis: summary of a workshop. Hepatology 44, 746-764 (2006); O'Mahony, C. A. & Vierling, J. M. Etiopathogenesis of primary sclerosing cholangitis. Semin Liver Dis 26, 3-21, doi:10.1055/s-2006-933559 (2006); Pollheimer, M. J., Trauner, M. & Fickert, P. Will we ever model PSC?—“it's hard to be a PSC model!”. Clin Res Hepatol Gastroenterol 35, 792-804, doi:S2210-7401(11)00151-3

[pii]10.1016/j.clinre.2011.04.014 (2011); Trauner, M. et al. New insights into autoimmune cholangitis through animal models. Dig Dis 28, 99-104, doi:000282072 [pii]10.1159/000282072 (2010); Vierling, J. M. Animal models for primary sclerosing cholangitis. Best Practice & Research Clinical Gastroenterology 15, 591-610 (2001)):

1.1. Reproducible onset and course of fibrous obliterative cholangitis.

1.2. Involvement of intra- and/or extra-hepatic ducts.

1.3. Atrophy of biliary epithelia.

1.4. Progression of microscopic and macroscopic lesions to ductopenia, portal fibrosis and biliary cirrhosis.

1.5. Association with gut inflammation, especially IBD/UC.

1.6. Immunological phenotypes of inflammatory cells infiltrating the portal tracts observed in human disease.

1.7. Spotty aberrant expression of increased MHC Class II and ICAM-1 by biliary epithelial cells.

1.8. Immunogenetic predisposition.

1.9. No immunosuppression, given the mechanism of action of aCD3 MAb.

2. A significant colony of well-characterized animals, and availability for a study.

3. Reproducible results that have been verified in more than one research group.

Existing models of sclerosing cholangitis can be divided into seven overlapping categories:

1. Enteric bacterial cell-wall components or colitis

2. Primary biliary epithelial cell (BEC) and endothelial cell injury

3. Chemically-induced cholangitis

4. Knockout mice

5. Cholangitis induced by infectious agents

6. Experimental biliary obstruction

7. Immune mediated cholangitis

I. Expert Opinions

Available models have been reviewed in several review papers published by PSC experts. All experts reviewing the field agree that there are no current available satisfactory animal models for PSC (O'Mahony, C. A. & Vierling, J. M. Etiopathogenesis of primary sclerosing cholangitis. Semin Liver Dis 26, 3-21, doi:10.1055/s-2006-933559 (2006); Pollheimer, M. J., Trauner, M. & Fickert, P. Will we ever model PSC?—“it's hard to be a PSC model!”. Clin Res Hepatol Gastroenterol 35, 792-804, doi:S2210-7401(11)00151-3 [pii]10.1016/j.clinre.2011.04.014 (2011); Trauner, M. et al. New insights into autoimmune cholangitis through animal models. Dig Dis 28, 99-104, doi:000282072 [pii]10.1159/000282072 (2010); Vierling, J. M. Animal models for primary sclerosing cholangitis. Best Practice & Research Clinical Gastroenterology 15, 591-610 (2001); LaRusso, N. F. et al. Primary sclerosing cholangitis: summary of a workshop. Hepatology 44, 746-764, doi:10.1002/hep.21337 (2006)). Although some existing models share some features with PSC and can be used to learn about certain stages of pathogenesis as well as to evaluate some treatments, none of those models is comprehensive enough to cover all aspects of PSC and is suitable for oral immunotherapy that functions by immune modulation.

In short, the following points need to be considered:

1. The models lack data on immunopathology, genetic predisposition, spotty aberrant BEC expression of increased MHC class II and ICAM-1, and progression of microscopic lesions to ductopenia, portal fibrosis and biliary epithelia.

2. Models that are associated with bile duct damage are usually due to mechanical obstruction or toxic effect and do not involve the immune mediated damage.

3. Models associated with immune derangement are mostly in immune-deficient mice and therefore are incompatible for the testing of aCD3 as an immunomodulatory agent.

4. Oral anti-CD3 treatment, which affects Tregs hence T effector cells, may be uninformative in these models which lack established evidence for the role of Tregs and/or effector lymphocytes in their pathogenesis.

II. Animal Models of PSC

1. Models involving enteric bacterial cell-wall components or colitis

1.1. Bacterial overgrowth of the small bowel

Surgical creation of self-filling loops of jejunum, creating bacterial overgrowth of native flora, is followed by hepatobiliary injury within 4-16 weeks in Lewis, Wistar and Sprague-Dawley rats (Lichtman, S. N., Keku, J., Clark, R. L., Schwab, J. H. & Sartor, R. B. Biliary tract disease in rats with experimental small bowel bacterial overgrowth. Hepatology 13, 766-772, doi:S0270913991001003 [pii] (1991)). In contrast, neither Fischer nor Buffalo rats developed injury after 16 weeks even though the loop sizes and the total anaerobic bacterial content of the loops were similar. The pathogenetic mechanisms of the model remain only partially understood, but this model provides evidence that bacterial cell wall components of anaerobic bacteria can induce Kupffer cell cytokine secretion in genetically susceptible rats, which results in some histopathological and cholangio-graphic features of PSC. Treated rats had irregular and dilated extrahepatic bile ducts with thickened walls (Lichtman, S. N., Wang, J. & Clark, R. L. A microcholangiographic study of liver disease models in rats. Academic Radiology 2, 515-521 (1995)).

The nature of genetic predisposition in this model remains unclear. Differences in MHC antigens do not explain susceptibility, since susceptible (Lewis) and non-susceptible (Fischer) rats are MHC-identical. This model is Kupffer-cell-mediated and not T-cell mediated. Hence oral anti-CD3 treatment, which affects Treg, may be uninformative in this model on the outcome of PSC treatment (Lichtman, S. N., Okoruwa, E. E., Keku, J., Schwab, J. H. & Sartor, R. B. Degradation of endogenous bacterial cell wall polymers by the muralytic enzyme mutanolysin prevents hepatobiliary injury in genetically susceptible rats with experimental intestinal bacterial overgrowth. The Journal of Clinical Investigation 90, 1313-1322 (1992)). Another shortcoming is that although initiated 20 years ago, this model has not been published or reproduced by researchers outside Lichtman's group. The relevance of this model to human PSC is unknown (Vierling, J. M. Animal models for primary sclerosing cholangitis. Best Practice & Research Clinical Gastroenterology 15, 591-610 (2001)).

1.2. Biliary sclerosis after IP injection of peptidoglycan-polysaccharide Rats injected once with a peptidoglycan-polysaccharide prepared from the cell wall of Streptococcus pyogenes develop hepatic granulomas and destructive arthritis, but no IBD or UC was detected (Cromartie, W. J., Craddock, J. G., Schwab, J. H., Anderle, S. & Yang, C. H. Arthritis in rats after systemic injection of streptococcal cells or cell walls. The Journal of experimental medicine 146, 1585-1602 (1977)). Adoptive transfer of T cells from injected rats to naïve rats produced arthritis, but liver pathology was not studied. Cholangiography showed focal strictures of intra-hepatic ducts and normal extra-hepatic ducts (Lichtman, S. N., Wang, J. & Clark, R. L. A microcholangiographic study of liver disease models in rats. Academic Radiology 2, 515-521 (1995)).

The immunological phenotype of inflammatory cells infiltrating the portal tract was not similar to PSC. Spotty aberrant BEC expression of increased MHC class II and ICAM-1 was not reported. There was no evidence for immunogenetic predisposition. This model presents only very limited aspects of PSC and is limited to the interhepatic bile duct; pathology is self-limited and mild relative to PSC. Liver involvement is unknown, without which this model is irrelevant to PSC. Mechanisms and role of genetic predisposition have not been studied. The role of Tregs in this model was not tested.

1.3. Granulomatous Colitis Induced with Muramyl Dipeptide

Muramyl dipeptide (MDP), a bacterial cell-wall fragment, causes granulomatous colitis in rabbits following emulsification with complete Freund's adjuvant and injection into the submucosa of the rectum and colon. Rabbits injected monthly for >9 months into 6 sites developed UC characterized by lymphocytic inflammation, granulomas, ulceration and epithelial regeneration. Only five of seven reported rabbits exhibited pericholangitis and periductal fibrosis, similar to early lesions in PSC (Kuroe, K. et al. Pericholangitis in a rabbit colitis model induced by injection of muramyl dipeptide emulsified with a long-chain fatty acid. Journal of gastroenterology 31, 347-352 (1996)).

This long-term model (>9 months) is not well established. The immunological phenotype of inflammatory cells infiltrating the portal tract was not similar to human disease. This model lacks data on immunopathology, Tregs, genetic predisposition, spotty aberrant BEC expression of increased MHC class II and ICAM-1, and progression of microscopic lesions to ductopenia, portal fibrosis and biliary epithelia. Liver function of all rabbits was normal throughout the experiment, such that this model is not relevant to the liver pathology of PSC.

1.4. Administration of N-formyl L-methionine L-leucine L-tyrosine (fMLT)

Daily administration of E. coli-derived fMLT+acetate to Wistar rat colon induced colitis and profound hepatic infiltration of mononuclear cells, mostly around small intrahepatic bile ducts (Yamada, S., Ishii, M., Liang, L. S., Yamamoto, T. & Toyota, T. Small duct cholangitis induced by N-formyl L-methionine L-leucine L-tyrosine in rats. J Gastroenterol 29, 631-636 (1994)). The preferential involvement of small bile ducts might be due to differences in blood supply between small and large bile ducts.

Data are missing on spotty aberrant BEC expression of increased MHC class II and ICAM-1 levels, ICAM and MHC II expression, immunopathology, genetic predisposition, Tregs, and progression of microscopic lesions to ductopenia, portal fibrosis and biliary epithelia. Neither periductal fibrosis nor obliterating cholangitis was observed in this model due to the short observation period, hence pathology unrelated to PSC. Acute life-threatening colitis due to intrarectal infusion results in very high mortality rates, which make the model very difficult for meaningful long-term studies even if relevant to PSC.

1.5. Dextran Sulfate Sodium (DSS) Treatment

Oral treatment of CD-1 mice with low-dose DSS induced chronic colitis accompanied by hepatobiliary lesions in 1/3 of mice, exhibiting portal inflammation and focal hepatocellular necrosis. No fibrosis typical of PSC developed within the one-month observation period (Nonomura, A., Kono, N., Minato, H. & Nakanuma, Y. Diffuse biliary tract involvement mimicking primary sclerosing cholangitis in an experimental model of chronic graft-versus-host disease in mice. Pathol Int 48, 421-427 (1998)).

This model is missing data on immunopathology, genetic predisposition, and spotty aberrant BEC expression of increased MHC class II and ICAM-1. There was no progression of microscopic lesions to ductopenia, portal fibrosis and biliary epithelia. This model may help elucidate a relationship between hepatic and colonic inflammation, yet is not associated with periductal fibrosis, hence irrelevant to PSC pathology.

2. Models of Primary Biliary Epithelial and Endothelial Cell Injury

2.1. Graft-Versus-Host Disease (GVHD)

Bile ducts are major targets in acute and chronic GVHD. The principal hepatic lesion in human GVHD and mouse models is non-suppurative destructive cholangitis mediated by T-cells and cytokines. Spleen and bone marrow cells of congenic B10.D2 mice were injected into sublethally irradiated BALB/c mice (Nonomura, A., Kono, N., Minato, H. & Nakanuma, Y. Diffuse biliary tract involvement mimicking primary sclerosing cholangitis in an experimental model of chronic graft-versus-host disease in mice. Pathol Int 48, 421-427 (1998)). Both intra- and extra-hepatic bile ducts were heavily involved in GVHD, showing features of non-suppurative cholangitis. Peak inflammation occurred 2-3 weeks post-transplantation, although reduced infiltration persisted during the full 14-month observation period. Distinct ductal and periductal fibrosis of intra- and extra-hepatic bile ducts was observed starting 1 week after transplantation and progressing for 2-3 months, which resembles only some aspects of PSC.

This model is missing data on spotty aberrant BEC expression of increased MHC class II and ICAM-1, role of Tregs, immunogenetic predisposition, and connection to gut inflammation. Findings of duct fibrosis have not been reported by others who have studied this GVHD model. This suggests that environmental factors may have caused the described periductal fibrosis and that this model is not reproducible or useful for preclinical studies. The GVHD model induces a multisystem disease that involves the skin and may affect other organs that are not affected in PSC. The somewhat similar liver lesions are not associated with the development of cirrhosis or severe fibrosis, nor with the changes in the extrahepatic bile ducts characteristic of PSC. The mice are also not fully immunocompetent. Thus, oral anti-CD3, which is an immunomodulatory agent, is not expected to have an effect.

2.2 Trinitrobenzene Sulphonic acid (TNBS) Infusion into the Portal Vein

TNBS binds to lysine residues in proteins and facilitates immune responses against these haptenated proteins. Following a single injection, Lewis rats exhibited a transient hepatic injury with elevated levels of serum aspartate aminotransferase, bilirubin and alkaline phosphatase that normalized in 15-30 days. Liver damage was accompanied by the production of ANCA mainly against catalase, as has been observed in PSC. Liver histology revealed very mild portal inflammation, ductular proliferation and only rarely some chronic cholangitis (Orth, T. et al. Anti-neutrophil cytoplasmic antibodies in a rat model of trinitrobenzenesulphonic acid-induced liver injury. Eur J Clin Invest 29, 929-939, doi:eci547 [pii] (1999)). However, there is no liver fibrosis, nor involvement of extra hepatic bile ducts.

Since the specificity for ANCA testing in PSC is low and the liver phenotype in TNBS-challenged rats is mild and reversible, the model does not mimic PSC. This model is missing data on spotty aberrant BEC expression of increased MHC class II and ICAM-1, and there was no apparent connection to gut inflammation.

2.3 Arterial and Capillary Plexus Injury

Intra-arterial infusions of floxuridine to dogs or rhesus monkeys caused fibrous inflammation and diffuse focal strictures. Lesions occurred commonly in both intra- and extra-hepatic bile ducts, but were sometimes restricted to one site (Dikengil, A. et al. Sclerosing cholangitis from intraarterial floxuridine. J Clin Gastroenterol 8, 690-693 (1986); Andrews, J. C. et al. Floxuridine-associated sclerosing cholangitis. A dog model. Invest Radiol 24, 47-51 (1989)). Experimental infusion of ethanol into hepatic arteries of monkeys resulted in diffuse focal strictures of intra-hepatic bile ducts, mild dilation of intervening segments of bile ducts, lymphocytic inflammation of the portal tracts, and portal fibrosis (Doppman, J. L. & Girton, M. E. Bile duct scarring following ethanol embolization of the hepatic artery: an experimental study in monkeys. Radiology 152, 621-626 (1984)). These findings suggest that direct injury of hepatic artery endothelia and branches causes periductal fibrous inflammation and focal strictures of both the intra-and extra-hepatic bile ducts. This model appears to be based on a toxic reaction.

This model is missing data on spotty aberrant BEC expression of increased MHC class II and ICAM-1, and on the role of Tregs, and there was no apparent connection to gut inflammation. This model is not practical since neither species is desirable for detailed studies of the pathogenetic mechanisms of fibrous inflammation and structuring. Available anti-CD3 antibodies are not reactive against the species used in this model. Data to date in the model are not statistically significant due to the use of few animals. The resulting disease was minor and did not mimic PSC in terms of pathology.

3. Chemically-Induced Cholangitis

3.1 Retrograde Biliary Injection of TNBS

Sprague-Dawley rats injected with TNBS by retrograde biliary administration developed histological and cholangiographic features resembling PSC (Mourelle, M., Salas, A., Vilaseca, J., Guarner, F. & Malagelada, J. R. Induction of chronic cholangitis in the rat by trinitrobenzenesulfonic acid. J Hepatol 22, 219-225, doi:0168-8278(95)80432-3 [pil] (1995)). TNBS haptenization was considered the principal mechanism for autoantibody production in this model. A one-year follow-up study after a single injection did not show morphological signs of PSC, indicating that multiple insults beyond TNBS may be needed to trigger chronic PSC (Mourelle, M., Salas, A., Vilaseca, J., Guarner, F. & Malagelada, J. R. Induction of chronic cholangitis in the rat by trinitrobenzenesulfonic acid. J Hepatol 22, 219-225, doi:0168-8278(95)80432-3 [pii] (1995)).

The damage observed is most likely due to a toxic effect, and is not directly involving the systemic immune system. This model features no apparent connection to gut inflammation and has no information about spotty aberrant BEC expression of increased MHC class II and ICAM-1. This model also has a very high mortality rate caused by the combined surgical/chemical trauma, which further limits its utility.

3.2. Feeding α-naphthylisothiocyanate (ANIT)

Feeding ANIT to Sprague-Dawley rats induced chronic cholangitis, with inflammatory infiltrates of the portal tracts by day 4, progression of portal inflammation by day 7, and extensive fibrosis by day 14 (Tjandra, K., Sharkey, K. A. & Swain, M. G. Progressive development of a Th1-type hepatic cytokine profile in rats with experimental cholangitis. Hepatology 31, 280-290, doi:S0270913900761702 [pii] 10.1002/hep.510310204 (2000)).

Although this model offers some resemblance to intrahepatic PSC, extrahepatic bile ducts remained normal throughout the experiments. Pathogenesis of induced cholangitis was not well studied. The acute nature of this model suggests that issues related to immunity are not involved in pathogenesis of this model. Other aspects of PSC not well characterized include immunogenetic predisposition, gut inflammation, spotty aberrant BEC expression of increased MHC class II and ICAM-1, and progression of microscopic lesions to ductopenia, portal fibrosis and biliary epithelia.

3.3. Feeding 3,5-diethoxycarbonyl-1, 4-dihydrocollidine (DDC)

Feeding DDC to Swiss albino mice led to increased biliary porphyrin secretion and induction of VCAM, osteopontin, TNFα expression in BEC, and minor microscopic features of PSC (Fickert, P. et al. A new xenobiotic-induced mouse model of sclerosing cholangitis and biliary fibrosis. Am J Pathol 171, 525-536, doi:S0002-9440(10)61986-4 [pii] 10.2353/ajpath.2007.061133 (2007)).

Bile duct plastination lacked beading and pruning of large ducts typical of PSC. Immunogenetic predisposition and gut inflammation are not well characterized. Although this model may be useful to study the complicated interplay among hepatocytes, BEC and mesenchymal cells in the pathogenesis of cholangiopathies and biliary fibrosis, the acute and non-chronic nature of this non-T-cell mediated model is distinct from PSC.

3.4. Feeding Lithocholic Acid (LCA)

LCA feeding is an increasingly used model for cholestatic liver injury. Short term (1-4 days) feeding LCA to Swiss albino mice led to bile infarcts, destructive cholangitis, periductal edema and fibrosis, resembling early stages of PSC (Fickert, P. et al. Lithocholic acid feeding induces segmental bile duct obstruction and destructive cholangitis in mice. Am J Pathol 168, 410-422, doi:S0002-9440(10)62102-5 [pii]10.2353/ajpath.2006.050404 (2006)). LCA feeding leads to periductal fibrosis via an efflux of bile fluids into the portal field and subsequent activation of BECs and periductal myofibroblasts. A role for effector T cells was not established in this model.

This model shares some features with PSC but is much more destructive to hepatocytes. Information is missing about this model, such as large duct morphology, consequences of long-term feeding, immunogenetic predisposition, and prevalence of spotty aberrant BEC expression of increased MHC class II and ICAM-1. As with other short-term models, the damage is not caused by an immune reaction and does not involve gut inflammation.

4. Knock-Out Models

4.1. Mdr2^{-/-} Mice

Mice with disruption of the Mdr2 gene encoding a canalicular phospholipid flippase (Mdr2^{-/-} mice) spontaneously develop cholangitis and onionskin-type periductal fibrosis mirroring some key features of PSC (Popov, Y., Patsenker, E., Fickert, P., Trauner, M. & Schuppan, D. Mdr2 (Abcb4)−/− mice spontaneously develop severe biliary fibrosis via massive dysregulation of pro- and antifibrogenic genes. Journal of hepatology 43, 1045-1054, doi:10.1016/j.jhep.2005.06.025 (2005)). These features are likely linked to the lack of biliary phospholipid secretion and consequently increased concentration of free non-micellar-bound bile acids, which subsequently cause biliary epithelial cell (BEC) damage. The role of T cells or other arms of the immune system were not established in this model.

The mechanism of disease induction does not include the induction of autoimmunity or immune effects and is not combined with IBD. It also lacks information about prevalence of spotty aberrant BEC expression of increased MHC class II and ICAM-1. Although this model may be useful for developing antifibrotic treatments against pathological results of PSC such as biliary fibrosis (Strack, I. et al. [beta]-Adrenoceptor blockade in sclerosing cholangitis of Mdr2 knockout mice: antifibrotic effects in a model of nonsinusoidal fibrosis. Lab Invest 91, 252-261, doi:http://www.nature.com/labinvest/journal/v91/n2/suppinfo/labinvest2010162s1.html (2011)), it does not represent the natural etiology of disease.

4.2. Cftr^{-/-} mice

Mice knocked out for the CF trans-membrane conductance regulator (Cftr^{-/-} mice) show conflicting results, which might be attributed to the age of the mice. One study described development of progressive liver disease with steatosis, focal cholangitis, inspissated bile and duct proliferation (Durie, P. R., Kent, G., Phillips, M. J. & Ackerley, C. A. Characteristic Multiorgan Pathology of Cystic Fibrosis in a Long-Living Cystic Fibrosis Transmembrane Regulator Knockout Murine Model. The American journal of pathology 164, 1481-1493, doi:10.1016/s0002-9440(10)63234-8 (2004)). Others reported an intestinal phenotype with no liver changes (Blanco, P. G. et al. Induction of colitis in cftr−/−mice results in bile duct injury. American Journal of Physiology-Gastrointestinal and Liver Physiology 287, G491-G496 (2004)).

Although this animal model demonstrates bile duct injury and shares some similarity to that seen in PSC, the histological changes do not meet the criteria for PSC since no fibrosis was present. Furthermore, CF mice are suffering from a severe combined disease, affecting multiple organs, bile ducts being one of them. The role of T cells or other arms of the immune system were not established in this model. The variability in performance of the model across different reports argues for unreliability.

4.3. Point Mutation in the Ferrochelatase Gene (fch)

Homozygous fch/fch mice develop cholangitis and severe biliary fibrosis, reflected in ductular proliferation, portal-portal bridging and progression to cirrhosis within a few months (Libbrecht, L. et al. Liver pathology and hepatocarcinogenesis in a long-term mouse model of erythropoietic protoporphyria.

The Journal of Pathology 199, 191-200, doi:10.1002/path.1257 (2003)). Variable amounts of protoporphyrin (PP) deposition were present in lumina of small bile ducts, resulting in incomplete obstruction. Liver disease is associated with the formation of bile with high concentrations of hydrophobic bile salts and PP with reduced cholesterol, phospholipid and glutathione content, which may cause BEC injury. The disease seems to involve mechanical obstruction, while the role of T cells or other arms of the immune system were not established. This model mirrors the DDC model and shares some pathologic features of PSC and is a reasonable model of EPP but not PSC. This model needs further clarification of the kinetics of bile duct system destruction Immunogenetic predisposition, gut inflammation and prevalence of spotty aberrant BEC expression of increased MHC class II and ICAM-1 are not well characterized.

5. Cholangitis Induced by Infectious Agents

5.1. Cryptosporidium Parvum (CP) Infection of Immunodeficient Mice

After CP infection, nu/nu immunodeficient mice showed severe cholangitis, pericholangitis and biliary fibrosis with portal-portal bridges, while SCID mice developed mild portal lymphocytic inflammation with spontaneous recovery. These findings suggest a crucial role for cell-mediated immunity in this model (Mead, J. R., Arrowood, M. J., Sidwell, R. W. & Healey, M. C. Chronic Cryptosporidium parvum Infections in Congenitally Immunodeficient SCID and Nude Mice. Journal of Infectious Diseases 163, 1297-1304, doi:10.1093/infdis/163.6.1297 (1991)). This model may be useful to determine the role of some cytokines and their receptors in secondary PSC. Biliary tract infections with CP may lead to PSC and biliary fibrosis in immunodeficient patients.

The main drawback is that the model employs immunodeficient mice, while oral anti-CD3 immunotherapy relies on immune modulation in a functional immune system.

5.2. Helicobacter Infection of Mice

Helicobacter species cause chronic infections of the GI tract and the bile duct system in humans and animals. IP injection of H. hepaticus induced focal necrosis and lymphocytic inflammation in susceptible (SCID/NCr, A/JCr and C3H/HeNCr) young mice, while older mice developed mild cholangitis and ductular proliferation. No fibrotic lesions were detected.

This model employs immunodeficient mice, while oral aCD3 immunotherapy relies on immune modulation in a functional immune system. Available studies do not provide information on the macroscopic appearance of the biliary system in order to judge potential relevance to PSC (Ward, J. M., Anver, M. R., Haines, D. C. & Benveniste, R. E. Chronic active hepatitis in mice caused by Helicobacter hepaticus. Am J Pathol 145, 959-968 (1994)). This model also lacks information about the prevalence of spotty aberrant BEC expression of increased MHC class II and ICAM-1.

6. Experimental Biliary Obstruction

Bile duct obstruction in rats and mice causes a deleterious sequence of events. Biliary pressure is immediately increased after bile duct ligation (BDL) and is accompanied by characteristic morphological features. Changes include hepatic necrosis, periportal inflammation and periductal edema of the bile ducts. These are followed by proliferative responses of BEC and hepatocytes. BDL induces liver fibrosis, showing increased levels of type I collagen, TIMP-1, and TGFI3 (Georgiev, P. et al. Characterization of time-related changes after experimental bile duct ligation. British Journal of Surgery 95, 646-656, doi:10.1002/bjs.6050 (2008)).

Like all other models involving mechanical damage to the biliary system, the model does not involve autoimmunity or the immune system, hence not informative. This model further lack information about immunogenetic predisposition and gut inflammation, and is not a model for testing of immunomodulatory agents.

7. Immune-Mediated Cholangitis

Young Wistar and Fisher mice were immunized with purified hyperplastic cholangiocytes from the same strain emulsified in Freund's complete adjuvant. One week post-dose-3, mice showed portal tract diffuse inflammation consisting predominantly of mononuclear cells as well as mononuclear infiltrate surrounding intrahepatic bile ducts.

Adoptive transfer of splenocytes from immunized mice led to non-supportive cholangitis within one week of administration. No MHC-II expression was observed in the bile duct. Immunization also elicited antibodies against cholangiocyte antigens.

Several features of this model contrast with known abnormalities in PSC. Inflammation is self-limiting and non-chronic, portal tract inflammation is mild, and there are no enzyme abnormalities (Ueno, Y., Phillips, J. O., Ludwig, J., Lichtman, S. N. & LaRusso, N. F. Development and characterization of a rodent model of immune-mediated cholangitis. Proceedings of the National Academy of Sciences 93, 216-220 (1996)). No fibrosis or cirrhosis is developed. No role for Tregs was described. Other missing information includes involvement of gut inflammation, involvement of outer hepatic ducts, prevalence of spotty aberrant BEC expression of increased MHC class II and ICAM-1, and progression of microscopic lesions to ductopenia, portal fibrosis and biliary epithelia.

III. Summary

Nine features of an animal model for PSC therapy with oral anti-CD3 MAb were described above. The literature was reviewed for available models with at least some desired attributes. Many models present some symptoms resembling PSC, yet do not mimic disease etiology, which is crucial for modeling PSC for oral anti-CD3 immunotherapy that functions through immune modulation. Other models employ immunodeficient animals not useful for immune-mediated therapy. Although various models have been studied, no single model has been established exhibiting even most attributes of PSC disease, which is genetically complex with unclear targets. A complete picture of PSC will need different models to study particular pathogenic steps of PSC. However, preclinical studies will not benefit from multiple models that mimic parts of pathogenesis since it will not create a complete picture of treatment efficacy for predicting clinical studies.

Thus, no existing animal model will be useful for a preclinical study of oral anti-CD3 immunotherapy.

### Example 2

### Treatment of PSC or PBC—Clinical Trial

There is no cure for chronic PSC or PBC, but some limited symptomatic treatments are available. Subjects may be treated with cholestyramine, which prevents reabsorption of bile. Inflammation may be managed by antibiotics. Liver transplantation is used for subjects with uncompensated cirrhosis as a result of the progress of the disease.

The efficacy of oral anti-CD3 immunotherapy is assessed in a clinical trial of patients with PSC or PBC. The subjects are treated daily during an interval of 6 months (180 days) with oral anti-CD3 at two dosage levels (1 mg and 5 mg) or with Placebo. It is noted that other dosing intervals and frequencies and dosage levels may be useful for treatment or preventing progression. Subjects would be evaluated for safety and efficacy parameters until Day 210.

Safety of anti-CD3 mAb is assessed by monitoring subjects for reported adverse events (AEs) by means of a subject diary and physical examinations and by interpreting the results of the various laboratory tests for safety, including general blood chemistry, liver and kidney functions (including bilirubin), and CBC including WBC differentials, as well as by comparing the frequency and patterns of AEs in the anti-CD3 treatment groups to that of the Placebo group. All safety tests are performed every two weeks for the first two months then monthly for the next five months through Day 210. In addition, CD3, CD4 and CD8 tests are performed by FACS on blood as measures of immunological safety; these tests are performed monthly through Day 210, and the results of the treatment groups are compared to those of the Placebo group.

Clinical efficacy is based on improvement in blood tests of inflammatory markers in the liver, especially levels of ALT, AST and bilirubin as specific markers of PSC or PBC, which will be tested monthly through Day 210, and endoscopic retrograde cholangio-pancreatography that will be done on Days 0 and 180. Fibro-scan would be performed on Days 0 and 180 in cases of cirrhosis (grades 1-2).

Immune modulation effects are evaluated on Days 0, 60, 120, 180 and 210. FACS analysis of T-cell markers may include but is not limited to CD4, CD8, CD25, LAP and

Foxp3. Serum cytokine analysis may include but is not limited to pro-inflammatory (IFN-γ, IL-1β, IL-2, IL-6, TNF-α) and anti-inflammatory (IL-4, IL-10, IL-13, IL-16, TGF-β) cytokines. In addition, serum anti-mitochondrial antibodies (AMA) are measured as a specific marker for PBC. For AMA-negative PBC subjects, anti-nuclear auto-antibodies (ANA) and/or anti-smooth-muscle auto-antibodies (SMA) are measured. The assessments for efficacy are ascertained for each subject by comparing values in efficacy parameters before anti-CD3 therapy to those during and after anti-CD3 therapy for each subject and then for each treatment group, as well as by comparing overall changes in efficacy parameters among one or more of the two anti-CD3 treatment groups compared to the placebo group.

### REFERENCES

1 Lee, Y. M. & Kaplan, M. M. Primary sclerosing cholangitis. New England Journal of Medicine 332, 924-933 (1995).
2 DAS, K. M. Immunopathogenesis of primary sclerosing cholangitis: possible role of a shared colonic and biliary epithelial antigen. Journal of Gastroenterology and Hepatology 19, S290-S294 (2004).
3 Brommé, U., Grunewald, J., Scheynius, A., Olerup, O. & Hultcrantz, R. Preferential Vβ3 usage by hepatic T lymphocytes in patients with primary sclerosing cholangitis. Journal of hepatology 26, 527-534, doi:10.1016/s0168-8278(97)80417-5 (1997).
4 Karlsen, T. H. et al. Genome-Wide Association Analysis in Primary Sclerosing Cholangitis. Gastroenterology 138, 1102-1111, doi:10.1053/j.gastro.2009.11.046 (2010).
5 La Russo, N. F. et al. Primary sclerosing cholangitis: summary of a workshop. Hepatology 44, 746-764 (2006).
6 O'Mahony, C. A. & Vierling, J. M. Etiopathogenesis of primary sclerosing cholangitis. Semin Liver Dis 26, 3-21, doi:10.1055/s-2006-933559 (2006).
7 Pollheimer, M. J., Trauner, M. & Fickert, P. Will we ever model PSC?—“it's hard to be a PSC model!”. Clin Res Hepatol Gastroenterol 35, 792-804, doi:S2210-7401(11)00151-3 [pii]10.1016/j.clinre.2011.04.014 (2011).
8 Trauner, M. et al. New insights into autoimmune cholangitis through animal models. Dig Dis 28, 99-104, doi:000282072 [pii]10.1159/000282072 (2010).
9 Vierling, J. M. Animal models for primary sclerosing cholangitis. Best Practice & Research Clinical Gastroenterology 15, 591-610 (2001).

10 Lichtman, S. N., Keku, J., Clark, R. L., Schwab, J. H. & Sartor, R. B. Biliary tract disease in rats with experimental small bowel bacterial overgrowth. Hepatology 13, 766-772, doi:S0270913991001003 [pii] (1991).
11 Lichtman, S. N., Wang, J. & Clark, R. L. A microcholangiographic study of liver disease models in rats. Academic Radiology 2, 515-521 (1995).
12 Lichtman, S. N., Okoruwa, E. E., Keku, J., Schwab, J. H. & Sartor, R. B. Degradation of endogenous bacterial cell wall polymers by the muralytic enzyme mutanolysin prevents hepatobiliary injury in genetically susceptible rats with experimental intestinal bacterial overgrowth. The Journal of Clinical Investigation 90, 1313-1322 (1992).
13 Cromartie, W. J., Craddock, J. G., Schwab, J. H., Anderle, S. & Yang, C. H. Arthritis in rats after systemic injection of streptococcal cells or cell walls. The Journal of experimental medicine 146, 1585-1602 (1977).
14 Kuroe, K. et al. Pericholangitis in a rabbit colitis model induced by injection of muramyl dipeptide emulsified with a long-chain fatty acid. Journal of gastroenterology 31, 347-352 (1996).
15 Yamada, S., Ishii, M., Liang, L. S., Yamamoto, T. & Toyota, T. Small duct cholangitis induced by N-formyl L-methionine L-leucine L-tyrosine in rats. J Gastroenterol 29, 631-636 (1994).
16 Nonomura, A., Kono, N., Minato, H. & Nakanuma, Y. Diffuse biliary tract involvement mimicking primary sclerosing cholangitis in an experimental model of chronic graft-versus-host disease in mice. Pathol Int 48, 421-427 (1998).
17 Orth, T. et al. Anti-neutrophil cytoplasmic antibodies in a rat model of trinitrobenzenesulphonic acid-induced liver injury. Eur J Clin Invest 29, 929-939, doi:eci547 [pii] (1999).
18 Dikengil, A. et al. Sclerosing cholangitis from intraarterial floxuridine. J Clin Gastroenterol 8, 690-693 (1986).
19 Andrews, J. C. et al. Floxuridine-associated sclerosing cholangitis. A dog model. Invest Radiol 24, 47-51 (1989).
220 Doppman, J. L. & Girton, M. E. Bile duct scarring following ethanol embolization of the hepatic artery: an experimental study in monkeys. Radiology 152, 621-626 (1984).
21 Mourelle, M., Salas, A., Vilaseca, J., Guarner, F. & Malagelada, J. R. Induction of chronic cholangitis in the rat by trinitrobenzenesulfonic acid. J Hepatol 22, 219-225, doi:0168-8278(95)80432-3 [pii] (1995).
22 Goetz, M., Lehr, H., Neurath, M., Galle, P. & Orth, T. Long-Term Evaluation of a Rat Model of Chronic Cholangitis Resembling Human Primary Sclerosing Cholangitis. Scandinavian journal of immunology 58, 533-540 (2003).
23 Tjandra, K., Sharkey, K. A. & Swain, M. G. Progressive development of a Th1-type hepatic cytokine profile in rats with experimental cholangitis. Hepatology 31, 280-290, doi:S0270913900761702 [pii]10.1002/hep.510310204 (2000).
24 Fickert, P. et al. A new xenobiotic-induced mouse model of sclerosing cholangitis and biliary fibrosis. Am J Pathol 171, 525-536, doi:S0002-9440(10)61986-4 [pii] 10.2353/ajpath.2007.061133 (2007).
25 Fickert, P. et al. Lithocholic acid feeding induces segmental bile duct obstruction and destructive cholangitis in mice. Am J Pathol 168, 410-422, doi:S0002-9440(10)62102-5 [pii]10.2353/ajpath.2006.050404 (2006).
26 Popov, Y., Patsenker, E., Fickert, P., Trauner, M. & Schuppan, D. Mdr2 (Abcb4)−/− mice spontaneously develop severe biliary fibrosis via massive dysregulation of pro- and antifibrogenic genes. Journal of hepatology 43, 1045-1054, doi:10.1016/j.jhep.2005.06.025 (2005).
27 Strack, I. et al. [beta]-Adrenoceptor blockade in sclerosing cholangitis of Mdr2 knockout mice: antifibrotic effects in a model of nonsinusoidal fibrosis. Lab Invest 91, 252-261, doi:http://www.nature.com/labinvest/journal/v91/n2/suppinfo/labinvest2010162s1.html (2011).
28 Durie, P. R., Kent, G., Phillips, M. J. & Ackerley, C. A. Characteristic Multiorgan Pathology of Cystic Fibrosis in a Long-Living Cystic Fibrosis Transmembrane Regulator Knockout Murine

Model. The American journal of pathology 164, 1481-1493, doi:10.1016/s0002-9440(10)63234-8 (2004).
29 Blanco, P. G. et al. Induction of colitis in cftr−/−mice results in bile duct injury. American Journal of Physiology-Gastrointestinal and Liver Physiology 287, G491-G496 (2004).
30 Libbrecht, L. et al. Liver pathology and hepatocarcinogenesis in a long-term mouse model of erythropoietic protoporphyria. The Journal of Pathology 199, 191-200, doi:10.1002/path.1257 (2003).
31 Mead, J. R., Arrowood, M. J., Sidwell, R. W. & Healey, M. C. Chronic Cryptosporidium parvum Infections in Congenitally Immunodeficient SCID and Nude Mice. Journal of Infectious Diseases 163, 1297-1304, doi:10.1093/infdis/163.6.1297 (1991).
32 Ward, J. M., Anver, M. R., Haines, D. C. & Benveniste, R. E. Chronic active hepatitis in mice caused by Helicobacter hepaticus. Am J Pathol 145, 959-968 (1994).
33 Georgiev, P. et al. Characterization of time-related changes after experimental bile duct ligation. British Journal of Surgery 95, 646-656, doi:10.1002/bjs.6050 (2008).
34 Ueno, Y., Phillips, J. 0., Ludwig, J., Lichtman, S. N. & LaRusso, N. F. Development and characterization of a rodent model of immune-mediated cholangitis. Proceedings of the National Academy of Sciences 93, 216-220 (1996).
35 La Russo, N. F. et al. Primary sclerosing cholangitis: summary of a workshop. Hepatology 44, 746-764, doi:10.1002/hep.21337 (2006).

### OTHER EMBODIMENTS

It is to be understood that while the invention has been described in conjunction with the detailed description thereof, the foregoing description is intended to illustrate and not limit the scope of the invention, which is defined by the scope of the appended claims Optionally any one or more embodiments, sub-embodiments and/or components of any embodiment may be combined. Also optionally any combination or subcombination of elements or embodiments may optionally be combined. Other aspects, advantages, and modifications are within the scope of the following claims.

1. A method of treating of PSC (primary sclerosing cholangitis) in a subject, comprising administering to the subject an anti-CD3 immune molecule orally or mucosally.

2. A method of preventing progression and/or delaying development of PSC (primary sclerosing cholangitis) in a subject, comprising administering to the subject an anti-CD3 immune molecule orally or mucosally.

3. The method of claim 1, wherein said PSC comprises PSC involving small and large ducts, and those involving the liver and the extra-hepatic ducts.

4. (canceled)

5. (canceled)

6. (canceled)

7. The method of claim 1, wherein said administering to the subject comprises administering a pharmaceutical composition comprising an anti-CD3 immune molecule suitable for oral or mucosal administration, in a dosage suitable for treatment of PSC.

8. The method of claim 1, wherein oral or mucosal administration comprises one or more of oral, pulmonary, buccal, nasal, intranasal, sublingual, rectal, or vaginal administration.

9. The method of claim 1, wherein said anti-CD3 immune molecule comprises an anti-CD3 antibody.

10. The method of claim 1, wherein said anti-CD3 antibody comprises a molecule selected from the group consisting of a whole antibody or active fragments thereof.

11. The method of claim 1, wherein the anti-CD3 antibody is selected from the group consisting of a murine mAb, a humanized mAb, a human mAb, and a chimeric mAb.

12. The method of claim 2, wherein said PSC comprises PSC involving small and large ducts, and those involving the liver and the extra-hepatic ducts.

13. The method of claim 2, wherein said administering to the subject comprises administering a pharmaceutical composition comprising an anti-CD3 immune molecule suitable for oral or mucosal administration, in a dosage suitable for preventing progression and/or delaying development of PSC.

14. The method of claim 2, wherein oral or mucosal administration comprises one or more of oral, pulmonary, buccal, nasal, intranasal, sublingual, rectal, or vaginal administration.

15. The method of claim 2, wherein said anti-CD3 immune molecule comprises an anti-CD3 antibody.

16. The method of claim 2, wherein said anti-CD3 antibody comprises a molecule selected from the group consisting of a whole antibody or active fragments thereof.

17. The method of claim 2, wherein the anti-CD3 antibody is selected from the group consisting of a murine mAb, a humanized mAb, a human mAb, and a chimeric mAb.
